(19) United States
(12) Patent Application Publication
(10) Pub. No.: US 2006/0253914 A1
(43) Pub. Date: Nov. 9, 2006

(54) Ha4,a new osteoblast-and chondrocyte-specific small secreted peptide, compositions and methods of use

(76) Inventors: Benoit De Crombrugghe, Houston, TX (US); Haruhiko Akiyama, Houston, TX (US)

Correspondence Address: FULBRIGHT & JAWORSKI L.L.P., 600 CONGRESS AVE., SUITE 2400, AUSTIN, TX, 78701, US

(21) Appl. No.: 10/534,099

(22) PCT Filed: Nov. 4, 2003
(86) PCT No.: PCT/US03/35139

§ 371 Date: Mar. 31, 2006

Related U.S. Application Data
(60) Provisional application No. 60/423,690, filed on Nov. 4, 2002.

(51) Int. Cl. A01K 67/027; C12Q 1/68; C07H 21/04; C12P 21/06; A61K 38/18; A61K 31/59; A61K 31/445; A61K 31/66; C07K 14/475

(52) U.S. Cl. 800/18; 514/12; 514/171; 435/6; 435/69.1; 435/320.1; 435/456; 435/458; 435/354; 530/350; 536/23.2; 530/388.25; 424/450; 514/319; 514/167; 514/102

(57) ABSTRACT

A polypeptide designated HA4, expressed selectively by bone tissue, is provided. Transgenic animals that fail to express HA4 have bone formation abnormalities reflective of osteoporosis, Also provided are methods for promoting bone formation by increasing the activity or expression of HA4, methods of diagnosing or treating bone degenerative diseases, and methods of screening for drugs that alter bone formation.

This application claims the priority of U.S. Provisional Patent Application No. 60/423,690, filed Nov. 4, 2002, the entire disclosure of which is specifically incorporated herein by reference. The government owns rights in the present invention pursuant to grant number PO1AR42919 from the National Institutes of Health.

### BACKGROUND OF THE INVENTION

1. Field of the Invention

The present invention relates generally to molecular mechanisms of bone formation. More specifically, the invention relates to to an osteoblast- and chondrocyte-specific small secreted peptide designated as HA4 that is involved in bone formation.

2. Description of Related Art

Bone formation is a carefully controlled developmental process involving morphogen-mediated patterning signals that define areas of initial mesenchyme condensation, followed by induction of cell-specific differentiation programs to produce chondrocytes and osteoblasts. Positional information is conveyed via gradients of molecules, such as Sonic Hedgehog, that are released from cells within a particular morphogenic field together with region-specific patterns of hox gene expression. These molecules in turn regulate the localized production of bone morphogenetic proteins and related molecules which initiate chondrocyte- and osteoblast-specific differentiation programs.

Differentiation requires the initial commitment of mesenchymal stem cells to a given lineage, followed by induction of tissue-specific patterns of gene expression. Considerable information about the control of osteoblast-specific gene expression has come from analysis of the promoter regions of genes encoding proteins like osteocalcin that are selectively expressed in bone. Both general and tissue-specific transcription factors control this promoter. Osf2/Cbfa1, the first osteoblast specific transcription factor to be identified, is expressed early in the osteoblast lineage and interacts with specific DNA sequences in the osteocalcin promoter essential for its selective expression in osteoblasts (Franceschi, 1999). Cbfa1 is needed for osteoblast differentiation.

The reduced bone mineral density (BMD) observed in osteoporosis results, in part, from reduced activity of bone-forming osteoblasts (Jackson, 2000). The identification of factors that participate in the cell differentiation process has been beneficial in developing treatment protocols for osteoporosis. However, it is likely that other factors participate in the differentiation process as well. Thus, it would be beneficial to identify these factors both for their use in diagnosis of bone degenerative disease and in their treatment.

### SUMMARY OF THE INVENTION

The present invention is drawn to HA4 polypeptides, as well as DNA segments encoding HA4 polypeptides. The present invention also provides methods of making such DNA segments and polypeptides, as well as their use in drug screening, diagnosis and therapy of bone disease. Antibodies and transgenic animals and cells relating to HA4 are disclosed as well.

Thus, in a particular aspect of the invention, there is provided a purified or a substantially purified HA4 protein or polypeptide. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition. In certain embodiments, the protein or polypeptide of the invention may be operatively linked to a second polypeptide sequence. It is also contemplated that purified or substantially purified peptides and polypeptides of between about 5 to 244 amino acids in length comprising a contiguous sequence from SEQ ID NO:2 are encompassed by the invention. Thus, for example the invention contemplates polypeptides or proteins of from about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 244 contiguous amino acids of SEQ ID NO:2

In another embodiment, an isolated nucleic acid segment encoding a polypeptide comprising the sequence as shown in SEQ ID NO:2 is provided. The nucleic acid segment may comprise the DNA sequence as shown in SEQ ID NO:1. The nucleic acid segment may further comprise a promoter operably linked to the region encoding the protein. The promoter may be an inducible promoter, a constitutive promoter or a tissue specific promoter. The tissue specific promoter may be a bone specific promoter. The nucleic acid segment may be comprised within a viral vector, such as an adenoviral vector, a retroviral vector, an adeno-associated viral vector, a vaccinia viral vector, a herpesviral vector or a pox viral vector. The nucleic acid segment may be comprised within a non-viral vector. The non-viral vector may be comprised in a lipid carrier. The nucleic acid segment may further comprise a region encoding a selectable marker protein.

Examples of constitutive viral promoters include the HSV, TK, RSV, LTR promoter sequence from retroviral vectors, SV40 and CMV promoters, of which the CMV promoter is a currently preferred example. Examples of constitutive mammalian promoters include various housekeeping gene promoters, as exemplified by the β actin promoter. Other promoters may be dectin-1, dectin-2, human CD11c, F4/80, SM22, RSV, SV40, Ad MLP, β-actin, MHC class I or MHC class II promoter,

Inducible promoters and/or regulatory elements are also contemplated for use with the expression vectors of the invention. Examples of suitable inducible promoters include promoters from genes such as cytochrome P450 genes, heat shock protein genes, metallothionein genes, hormone-inducible genes, such as the estrogen gene promoter, and such like. Promoters that are activated in response to exposure to ionizing radiation, such as fos, jun and egr-1, are also contemplated.

Tissue-specific promoters and/or regulatory elements will be particularly useful in certain embodiments. Osteoblast-specific promoters that will be used are the 2.3 kB promoter of the mouse gene for pro-α1(I)collagen and the 1.1 kB mouse osteocalcin promoter.

The nucleic acid segment also may be characterized as (a) a nucleic acid segment comprising a sequence region that consists of 14 nucleotides that have the same sequence as, or complementary to, at least 14 contiguous nucleotides of SEQ ID NO:1; or (b) a nucleic acid segment of from 14 to 10,000 nucleotides in length that hybridizes to the nucleic acid segment of SEQ ID NO:1, or the complement thereof, under stringent hybridization conditions. The segment may comprise a sequence region of at least 14, 17, 20, 25 or 30 contiguous nucleotides from SEQ ID NO:1 or the complement thereof. The segment may be 17, 20, 25 or 30 nucleotides in length.

Nucleic acids of the invention may also be operatively linked to other protein-encoding nucleic acid sequences. This will generally result in the production of a fusion protein following expression of such a nucleic acid construct. Both N-terminal and C-terminal fusion proteins are contemplated. Virtually any protein- or polypeptide-encoding DNA sequence, or combinations thereof, may be fused to an HA4 sequence in order to encode a fusion protein. This includes DNA sequences that encode targeting polypeptides, therapeutic proteins, proteins for recombinant expression, proteins to which one or more targeting polypeptides is attached, protein subunits and the like.

The invention further includes DNA segments comprising the 5′ untranslated regions (5′ UTR) and 3′ UTR of HA4 genomic DNA, and 5′-flanking regions and 3′-flanking regions of HA4, including those that regulate HA4 expression. The inventors contemplate experiments wherein an isolated promoter fragment of the HA4 gene will be used to drive transcription of a reporter gene such as the luciferase gene in recombinant cells or transgenic mice. Thus, in one aspect of the invention, a DNA segment comprising the 5′-flanking regions of HA4 operatively linked to a heterologous gene or a DNA segment that encodes a selected protein (e.g., a screenable marker) are contemplated. Osteoblast promoters may be used to obtain targeted expression of a gene in osteoblasts.

Vectors and plasmids may be constructed with at least one multiple cloning site. In certain embodiments, the expression vector will comprise a multiple cloning site that is operatively positioned between a promoter and an HA4 gene sequence. Such vectors may be used, in addition to their uses in other embodiments, to create N-terminal fusion proteins by cloning a second protein-encoding DNA segment into the multiple cloning site so that it is contiguous and in-frame with the HA4 sequence.

In other embodiments, expression vectors may comprise a multiple cloning site that is operatively positioned downstream from the expressible HA4 gene sequence. These vectors are useful, in addition to other uses, in creating C-terminal fusion proteins by cloning a second protein-encoding DNA segment into the multiple cloning site so that it is contiguous and in-frame with the HA4 sequence. Vectors and plasmids in which a second protein- or RNA-encoding nucleic acid segment is also present are, of course, also encompassed by the invention, irrespective of the nature of the nucleic acid segment itself. Expression vectors may also contain other nucleic acid sequences, such as IRES elements, polyadenylation signals, splice donor/splice acceptor signals, and the like.

Particular examples of suitable expression vectors are those adapted for expression using a recombinant adenoviral, recombinant adeno-associated viral (AAV) or recombinant retroviral system. Vaccinia virus, herpes simplex virus, cytomegalovirus, and defective hepatitis B viruses, amongst others, may also be used.

Recombinant host cells form another aspect of the present invention. Such host cells will generally comprise at least one copy of an isolated HA4 gene linked to a heterologous promoter. Preferred cells for expression purposes will be prokaryotic host cells or eukaryotic host cells. Accordingly, cells such as bacterial, yeast, fungal, insect, nematode and plant cells are also possible. An example of a preferred bacterial host cell is E. coli. Examples of suitable eukaryotic host cells include VERO cells, HeLa cells, cells of Chinese hamster ovary (CHO) cell lines, COS cells, such as COS-7, and W138, BHK, HepG2, 3T3, RIN, MDCK, A549, PC12, K562 and 293 cells. Cells also include transgenic cells derived from transgenic animals engineered to overexpress or not express HA4, or to express a screenable marker under the control of HA4 regulatory signals. The marker may be luciferase, green fluorescent protein or any other gene whose expression is readily detected.

Many methods of using HA4 genes are obtained from the present invention, such as expressing an HA4 protein in a cell. More specific methods obtained from the invention are methods for identifying a modulatory agent that inhibits, stimulates, or modulates the expression of HA4. Thus, provided is a method for identifying a modulator of HA4 transcription, the method comprising admixing (i) a cell expressing HA4 or a cell with a reporter gene operably linked to an HA4 promoter, and (ii) a candidate substance. A candidate substance that alters the transcription of the HA4 gene or reporter gene is a modulator.

The invention also provides methods for identifying a bone cell stimulatory agent, comprising the steps of (a) admixing a composition comprising a population of precursor cells capable of expressing HA4; (b) incubating the admixture with a candidate substance; (c) testing the admixture for precursor cell differentiation; and (d) identifying the candidate substance that stimulates the differentiation of precursor cells into osteoblasts. In some embodiments, the precursor cell may be a mesenchymal precursor cell. The assay may be modified such that the precursor cells are stimulated to differentiate into osteoblasts, and the candidate substance is monitored for its ability to inhibit this process.

Agents that modulate HA4 expression and/or activity may be used to treat a number of bone-related diseases, such as osteoporosis, glucocorticoid induced osteoporosis, Paget's disease, abnormally increased bone turnover, periodontal disease, tooth loss, bone fractures, rheumatoid arthritis, periprosthetic osteolysis, osteogenesis imperfecta, metastatic bone disease, hypercalcemia of malignancy and the like. The inventors contemplate that HA4 proteins and HA4 expression constructs will increase HA4 expression and activity, whereas HA4 antisense constructs, ribozymes and single-chain antibodies will inhibit HA4 expression and/or activity.

Additionally, the present invention provides for a non-human transgenic animal, cells of which comprise one allele of the HA4 gene that does not express a functional HA4 product. The non-human transgenic animal may be a mouse. The non-human transgenic animal may alternatively have cells which comprise an expression cassette comprising an HA4 5′-regulatory region operably linked to a screenable marker gene. The screenable marker gene is luciferase, green fluorescent protein, and β-galactosidase.

Following longstanding patent law convention, the word “a” and “an,” when used in conjunction with the word comprising, mean “one or more” in this specification, including the claims. Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Genomic Structure of HA4 Genes.

FIG. 2—Northern Blot Analysis of HA4 Expression in Adult Mouse Tissues.

FIG. 3—Northern Blot Analysis of HA4 Expression During Mouse Embryogenesis.

FIG. 4—In situ Hybridization of HA4.

FIG. 5—X-gal Staining of HA4 Heterozygous Embryos.

FIG. 6—X-gal Staining of HA4 a Heterozygous Embryo.

FIG. 7—HA4 deficient mice have reduced bone density and provide a mouse model for human osteoporosis.

FIG. 8—Generation of transgenic mice and detection of HA4 protein in serum.

FIG. 9—Production of recombinant HA4 protein.

### DETAILED DESCRIPTION OF THE ILLUSTRATIVE EMBODIMENTS

Bone formation is a complex process that involves the differentiation of mesenchymal cell precursors into osteoblasts. It is believed that defects in this process can lead to various diseases, including those classified as degenerative bone diseases. The most important of these diseases is osteoporosis, literally meaning a disease of too little bone, which results in fragility fractures that occur with very little trauma. It is becoming progressively more common, partly because it is a disease that increases in frequency in patients who are over 60 years of age, a segment of the population that is progressively increasing. Osteoporosis is much more common in elderly females than in elderly males because the estrogen deficiency that occurs at the time of menopause leads to increased bone destruction, which is not compensated by a corresponding increase in bone formation (i.e., a negative bone balance), resulting in bone loss and, eventually, osteoporosis in many females.

The inventors identified a cDNA encoding a small secreted polypeptide containing a collagen triple helix repeat, designated as HA4, using a suppression subtraction between BMP-untreated and BMP-treated chondrogenic ATDC5 cells. In newborn homozygous HA4 mutants, reduced bone density was observed, and the number of bone trabecules was markedly reduced. This phenotype mimics that observed in humans with osteoporosis. The inventors thus have demonstrated a role for HA4 in bone and cartilage metabolism.

### I. HA4 POLYPEPTIDES

A. Polyepeptides and Peptides

As used herein below, the term HA4 should be interpreted to include not only the HA4 polypeptide of 244 amino acids, but also glycosylated forms as well as non-glycosylated forms of the molecule, and other members of the HA4 family. The present invention also encompasses peptides of about 3 to about 50 amino acids, and polypeptides of greater than 50 amino acids. All the “proteinaceous” terms described above may be used interchangeably herein.

In certain embodiments the size of the at least one proteinaceous molecule may comprise, but is not limited to, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, to about 244 reisdues. Fusion of greater size also are contemplated.

As used herein, an “amino acid molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties. Accordingly, the term “proteinaceous composition” encompasses amino molecule sequences comprising at least one of the 20 common amino acids in naturally synthesized proteins, or at least one modified or unusual amino acid, including but not limited to those shown on Table 1 below.

TABLE 1
Modified and Unusual Amino Acids

| Abbr. | Amino Acid |
|---|---|
| Aad | 2-Aminoadipic acid |
| Baad | 3-Aminoadipic acid |
| Bala | β-alanine, β-Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| Baib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| AHyl | allo-Hydroxylysine |
| 3Hyp | 3-Hydroxyproline |
| 4Hyp | 4-Hydroxyproline |
| Ide | Isodesmosine |
| AIle | allo-Isoleucine |
| MeGly | N-Methylglycine, sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

In further embodiments, the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

B. Purification of HA4 Proteins

Further aspects of the present invention concern the purification of an HA4 protein or polypeptide. The term “purified protein” as used herein, is intended to refer to an HA4 composition isolatable from natural sources such as osteoblastic MC3T3-E1 cells and undifferentiated ATDC5 cells, or recombinant host cells, wherein the HA4 is purified to any degree relative to its naturally-obtainable state. It is contemplated that the purified HA4 proteins or polypeptides of the invention will generally possess HA4 activity. That is, they will have the capacity to promote osteoblast differentiation and/or bone formation.

HA4 may be purified from extracts of various cells by immunoprecipitation using polyclonal anti-HA4 antibodies or monoclonal antibodies (MAb) (see below). In one embodiment, a cDNA encoding HA4 is expressed in a host cell, such as bacteria, yeast cells, insect cells, or mammalian cells, and the expressed proteins purified using antibodies against HA4.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite, lectin affinity and other affinity chromatography steps; isoelectric focusing; gel electrophoresis; and combinations of such and other techniques. A specific example is the purification of HA4 using immunoprecipitation with anti-HA4 antibodies.

Where the term “substantially purified” is used, this will refer to a composition in which HA4 forms the major component of the composition, such as constituting about 50% of the proteins in the composition or more. In preferred embodiments, a substantially purified protein will constitute more than 60%, 70%, 80%, 90%, 95% or 99% of the proteins in the composition.

A polypeptide or protein that is “purified to homogeneity,” as applied to the present invention, means that the polypeptide or protein has a level of purity where the polypeptide or protein is substantially free from other proteins and biological components. For example, a purified polypeptide or protein will often be sufficiently free of other protein components so that degradative sequencing may be performed successfully.

Various methods for quantifying the degree of purification of the HA4 protein will be known to those of skill in the art in light of the present disclosure. These include, for example, determining the specific activity of an active fraction, or assessing the number of polypeptides within a fraction by gel electrophoresis. Assessing the number of polypeptides within a fraction by SDS/PAGE analysis will often be preferred in the context of the present invention, e.g., in assessing protein purity.

As mentioned above, although preferred for use in certain embodiments, there is no general requirement that the HA4 proteins or polypeptides always be provided in their most purified state. Indeed, it is contemplated that less substantially purified proteins or polypeptides, which are nonetheless enriched in HA4 activity relative to the natural state, will have utility in certain embodiments. For example, less purified HA4 preparations may contain molecules that are associated naturally with HA4. If so, this may, ultimately, lead to the identification of unique molecules that associate with HA4 on the cell surfaces (e.g., co-receptors) or in the cytoplasma (e.g., signaling components).

Methods exhibiting a lower degree of relative purification may have advantages in total recovery of protein product, or in maintaining the activity of an expressed protein. Inactive products also have utility in certain embodiments, such as, e.g., in antibody generation.

Partially purified HA4 fractions for use in such embodiments may be obtained by subjecting cells or cell extracts to one or a combination of the steps described. Substituting certain steps with improved equivalents is also contemplated to be useful. For example, it is appreciated that a cation-exchange column chromatography performed utilizing an HPLC apparatus will generally result in a greater “-fold” purification than the same technique utilizing a low pressure chromatography system.

C. Biologically Functional Equivalents and Structural Equivalents

Modifications may be made in the structure of HA4 and still obtain a molecule having like or otherwise desirable characteristics. For example, certain amino acids may be substituted for other amino acids in a protein structure without appreciable loss of interactive binding capacity with structures such as, for example, antigen-binding regions of antibodies or binding sites on substrate molecules, receptors, or osteoblasts. Since it is the interactive capacity and nature of a protein that defines that protein's biological functional activity, certain amino acid sequence substitutions can be made in a protein sequence (or, of course, its underlying DNA coding sequence) and nevertheless obtain a protein with like (agonistic) properties. Equally, the same considerations may be employed to create a protein or polypeptide with countervailing (e.g., antagonistic) properties. It is thus contemplated by the inventors that various changes may be made in the sequence of HA4 protein or polypeptide (or underlying DNA) without appreciable loss of their biological utility or activity.

In terms of functional equivalents, it is also well understood by the skilled artisan that, inherent in the definition of a biologically functional equivalent protein or polypeptide, is the concept that there is a limit to the number of changes that may be made within a defined portion of the molecule and still result in a molecule with an acceptable level of equivalent biological activity. Biologically functional equivalent polypeptides are thus defined herein as those polypeptides in which certain, not most or all, of the amino acids may be substituted. Of course, a plurality of distinct proteins/polypeptides with different substitutions may be made and used in accordance with the invention.

It is also well understood that where certain residues are shown to be particularly important to the biological or structural properties of a protein or polypeptide, e.g., residues in active sites, such residues may not generally be exchanged. Amino acid substitutions are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine; and phenylalanine, tryptophan and tyrosine; are defined herein as biologically functional equivalents.

Conservative substitutions well known in the art include, for example, the changes of alanine to serine; arginine to lysine; asparagine to glutamine or histidine; aspartate to glutamate; cysteine to serine; glutamine to asparagine; glutamate to aspartate; glycogen to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte and Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity, particularly where the biological functional equivalent protein or polypeptide thereby created is intended for use in immunological embodiments, as in the present case. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e., with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±0.5 are even more particularly preferred.

While discussion has focused on functionally equivalent polypeptides arising from amino acid changes, it will be appreciated that these changes may be effected by alteration of the encoding DNA; taking into consideration also that the genetic code is degenerate and that two or more codons may code for the same amino acid. A table of amino acids and their codons is presented herein for use in such embodiments, as well as for other uses, such as in the design of probes and primers and the like.

Polypeptides corresponding to one or more antigenic determinants, or “epitopic core regions,” of HA4 can also be prepared. Such polypeptides should generally be at least five or six amino acid residues in length, and may contain up to about 35-50 residues or so. While peptides can be created by proteolytic cleavage, a more typical methods is to synthesize the peptides. Synthetic polypeptides will generally be about 35 residues long, which is the approximate upper length limit of automated polypeptide synthesis machines, such as those available from Applied Biosystems (Foster City, Calif.). Longer polypeptides may also be prepared, e.g., by recombinant means.

U.S. Pat. No. 4,554,101 (Hopp, incorporated herein by reference) teaches the identification and preparation of epitopes from primary amino acid sequences on the basis of hydrophilicity. Through the methods disclosed in Hopp, one of skill in the art would be able to identify epitopes from within an amino acid sequence. Numerous scientific publications have also been devoted to the prediction of secondary structure, and to the identification of epitopes, from analyses of amino acid sequences (Chou and Fasman, 1974a,b; 1978a,b; 1979). Any of these may be used, if desired, to supplement the teachings of Hopp in U.S. Pat. No. 4,554,101. Moreover, computer programs are currently available to assist with predicting antigenic portions and epitopic core regions of proteins. Examples include those programs based upon the Jameson-Wolf analysis (Jameson and Wolf, 1988; Wolf et al., 1988), the program PepPlot® (Brutlag et al., 1990; Weinberger et al., 1985), and other new programs for protein tertiary structure prediction (Fetrow and Bryant, 1993). Further commercially available software capable of carrying out such analyses is termed MacVector (IBI, New Haven, Conn.).

In further embodiments, major antigenic determinants of a polypeptide may be identified by an empirical approach in which portions of the gene encoding the polypeptide are expressed in a recombinant host, and the resulting proteins tested for their ability to elicit an immune response. For example, PCR™ can be used to prepare a range of polypeptides lacking successively longer fragments of the C-terminus of the protein. The immunoactivity of each of these polypeptides is determined to identify those fragments or domains of the polypeptide that are immunodominant. Further studies in which only a small number of amino acids are removed at each iteration then allows the location of the antigenic determinants of the polypeptide to be more precisely determined.

Once one or more such analyses are completed, polypeptides are prepared that contain at least the essential features of one or more antigenic determinants. The polypeptides are then employed in the generation of antisera against the polypeptide. Minigenes or gene fusions encoding these determinants can also be constructed and inserted into expression vectors by standard methods, for example, using PCR™ cloning methodology.

In addition to the peptidyl compounds described herein, the inventors also contemplate that other sterically similar compounds may be formulated to mimic the key portions of the polypeptide structure. Such compounds, which may be termed peptidomimetics, may be used in the same manner as the polypeptides of the invention and hence are also functional equivalents.

Certain mimetics that mimic elements of protein secondary structure are described in Johnson et al. (1993). The underlying rationale behind the use of polypeptide mimetics is that the polypeptide backbone of proteins exists chiefly to orientate amino acid side chains in such a way as to facilitate molecular interactions, such as those of antibody and antigen. A polypeptide mimetic is thus designed to permit molecular interactions similar to the natural molecule.

Some successful applications of the polypeptide mimetic concept have focused on mimetics of β-turns within proteins, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

The generation of further structural equivalents or mimetics may be achieved by the techniques of modeling and chemical design known to those of skill in the art. The art of receptor modeling is now well known, and by such methods a chemical that binds to the osteoblast HA4 receptor can be designed and then synthesized. It will be understood that all such sterically similar constructs fall within the scope of the present invention.

D. Production of Antibodies Against HA4

Means for preparing and characterizing antibodies are well known in the art (see, e.g., Antibodies: A Laboratory Manual, Cold Spring Harbor Laboratory, 1988; incorporated herein by reference). The methods for generating monoclonal antibodies (MAbs) generally begin along the same lines as those for preparing polyclonal antibodies. Briefly, a polyclonal antibody is prepared by immunizing an animal with an immunogenic composition in accordance with the present invention (either with or without prior immunotolerizing, depending on the antigen composition and protocol being employed) and collecting antisera from that immunized animal.

A wide range of animal species can be used for the production of antisera. Typically the animal used for production of anti-antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, μ-maleimidobencoyl-N-hydroxysuccininide ester, carbodiimyde and bis-biazotized benzidine.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, toxins or synthetic compositions.

Adjuvants that may be used include IL-1, IL-2, IL-4, IL-7, IL-12, y-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion. MHC antigens may even be used.

Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polygonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified HA4 protein, polypeptide or peptide (or any osteoblast composition, if used after tolerization to common antigens). The immunizing composition is administered in a manner effective to stimulate antibody producing cells.

The methods for generating MAbs generally begin along the same lines as those for preparing polyclonal antibodies. Rodents such as mice and rats are preferred animals, however, the use of rabbit, sheep or frog cells is also possible. The use of rats may provide certain advantages (Goding, 1986), but mice are preferred, with the BALB/c mouse being most preferred as this is most routinely used and generally gives a higher percentage of stable fusions. The inventors have generated the MAb against mouse HA4 in rats. This was primarily because it is technically difficult to immune mice with molecules of mouse origin. On the other hand, the inventors will prefer mice for the generation of MAb against human HA4.

The animals are injected with antigen, generally as described above. The antigen may be coupled to carrier molecules such as keyhole limpet hemocyanin if necessary. The antigen would, typically be mixed with adjuvant, such as Freund's complete or incomplete adjuvant. Booster injections with the same antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is accessible.

Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XXO Bul; for rats, one may use R210.RCY3, Y3-Ag 1.2.3, IR983F and 4B210; and U-266, GM1500-GRG2, LICR-LON-HMy2 and UC729-6 all of which are useful in connection with human cell fusions. One preferred murine myeloma cell is the NS-1 myeloma cell line (also termed P3-NS-1-Ag4-1), which is readily available from the NIGMS Human Genetic Mutant cell Repository by requesting cell line repository number GM3573. Another mouse myeloma cell line that may be used is the 8-azaguanine-resistant mouse murine myeloma SP2/0 non-producer cell line.

### II. DNA AND RNA SEGMENTS FOR HA4

A. DNA Segments

Important aspects of the present invention concern isolated DNA segments and recombinant vectors encoding HA4, and the creation and use of recombinant host cells that express HA4 through the application of DNA technology. More specifically, the present invention concerns mammalian DNA segments, isolated away from other mammalian genomic DNA segments or total chromosomes. Preferred sources for the HA4 DNA segments of the invention are human gene sequences. In cloning an HA4 sequence of the invention, one may advantageously choose an established osteoblast line. But other sources will be equally appropriate, such as cDNA or genomic libraries. The DNA segments of the invention are capable of conferring HA4-like activity or properties, such as defined herein below, to a recombinant host cell when incorporated into the recombinant host cell.

As used herein, the term “DNA segment” refers to a DNA molecule that has been isolated substantially free of total genomic DNA and chromosomes of a particular species. Therefore, a DNA segment encoding HA4 refers to a DNA segment that contains HA4 coding sequences yet is isolated away from, or purified free from, total genomic DNA of tissues known to contain relatively large numbers of osteoblasts, or of the BMP2-treated C2C12 line.

A DNA segment comprising an isolated or purified HA4 gene also refers to a DNA segment including HA4 coding sequences and, in certain aspects, regulatory sequences, isolated substantially away from other naturally occurring genes or protein encoding sequences. In this respect, the term “gene” is used for simplicity to refer to a DNA segment that encodes a polypeptide or a functional protein. As will be understood by those in the art, this functional term includes both genomic sequences, cDNA sequences and smaller engineered gene segments that express, or may be adapted to express, proteins, polypeptides or peptides.

“Isolated substantially away from other coding sequences” means that the gene of interest, in this case HA4, forms the significant part of the coding region of the DNA segment, and that the DNA segment does not contain large portions of naturally-occurring coding DNA, such as large chromosomal fragments or other functional genes or cDNA coding regions. Of course, this refers to the DNA segment as originally isolated, and does not exclude genes or coding regions later added to the segment by the hand of man.

In particular embodiments, the invention concerns isolated DNA segments and recombinant vectors incorporating DNA sequences that encode an HA4 protein or polypeptide that includes within its amino acid sequence an amino acid sequence in accordance with SEQ ID NO:2, corresponding to human or mammalian HA4.

In certain embodiments, the invention concerns isolated DNA segments and recombinant vectors that encode a protein or polypeptide that includes within its amino acid sequence an amino acid sequence essentially as set forth in SEQ ID NO:2. Naturally, where the DNA segment or vector encodes a full length HA4 protein, or is intended for use in expressing the HA4 protein, the most preferred sequences are those that are essentially as set forth in SEQ ID NO:2.

The term “a sequence essentially as set forth in SEQ ID NO:2” means that the sequence substantially corresponds to a portion of SEQ ID NO:2 and has relatively few amino acids that are not identical to, or a biologically functional equivalent of, the amino acids of SEQ ID NO: 2. The term “biologically functional equivalent” is well understood in the art and is further defined in detail herein. Accordingly, sequences that have between about 70% and about 80%; or more preferably, between about 81% and about 90%; or even more preferably, between about 91% and about 99%; of amino acids that are identical or functionally equivalent to the amino acids of SEQ ID NO:2 will be sequences that are “essentially as set forth in SEQ ID NO:2.”

In other embodiments, the invention concerns isolated DNA segments and recombinant vectors that include within their sequence a nucleic acid sequence essentially as set forth in SEQ ID NO:1. The term “essentially as set forth in SEQ ID NO:1” is used in the same sense as described above and means that the nucleic acid sequence substantially corresponds to a portion of SEQ ID NO:1 and has relatively few codons that are not identical, or functionally equivalent, to the codons of SEQ ID NO:1. The term “functionally equivalent codon” is used herein to refer to codons that encode the same amino acid, such as the six codons for arginine or serine, and also refers to codons that encode biologically equivalent amino acids. Table 1 sets forth the amino acids and codons which encode each amino acid.

TABLE 2

| Amino Acids | Codons | | | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

It is within the scope of the invention in certain aspects that high level protein production may be achieved by reducing criteria for osteoblast differentiation. In certain embodiments it is within the invention to produce proteins lacking activity. Such proteins might be useful in very high volume to raise antibodies to the protein.

It will also be understood that amino acid and nucleic acid sequences may include additional residues, such as additional N— or C-terminal amino acids or 5′ or 3′ sequences, and yet still be essentially as set forth in one of the sequences disclosed herein, so long as the sequence meets the criteria set forth above, including the maintenance of osteoblast differentiation activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

Suitable high stringency hybridization conditions will be well known to those of skill in the art and are clearly set forth herein, for example conditions such as relatively low salt and/or high temperature conditions, such as provided by 0.02M-0.15M NaCl at temperatures of 50° C. to 70° C., for applications requiring high selectivity. Such relatively stringent conditions tolerate little, if any, mismatch between the probe and the template or target strand, and would be particularly suitable for isolating HA4 genes.

Naturally, the present invention also encompasses DNA segments that are complementary, or essentially complementary, to the sequence set forth in SEQ ID NO:1. Nucleic acid sequences that are “complementary” are those that are capable of base-pairing according to the standard Watson-Crick complementary rules. That is, that the larger purines will always base pair with the smaller pyrimidines to form only combinations of guanine paired with cytosine (G:C) and adenine paired with either thymine (A:T), in the case of DNA, or adenine paired with uracil (A:U) in the case of RNA.

As used herein, the term “complementary sequences” means nucleic acid sequences that are substantially complementary, as may be assessed by the same nucleotide comparison set forth above, or as defined as being capable of hybridizing to the nucleic acid segment of SEQ ID NO:1 under relatively stringent conditions such as those described herein. As such, these complementary sequences are substantially complementary over their entire length and have very few base mismatches. For example, nucleic acid sequences of six bases in length may be termed complementary when they hybridize at five out of six positions with only a single mismatch. Naturally, nucleic acid sequences which are “completely complementary” will be nucleic acid sequences which are entirely complementary throughout their entire length and have no base mismatches. Equivalents will show transcriptional activity. This is one feature which will distinguish it from non-HA4 nucleic acid sequences.

Antisense constructs are oligo- or polynucleotides comprising complementary nucleotides to the coding segment of a DNA molecule, such as a gene or cDNA, including both the exons, introns and exon:intron boundaries of a gene. Antisense molecules are designed to inhibit the transcription, translation or both, of a given gene or construct, such that the levels of the resultant protein product are reduced or diminished. Antisense RNA constructs, or DNA encoding such antisense RNAs, may be employed to inhibit gene transcription or translation or both within a host cell, either in vitro or in vivo, such as within a host animal, including a human subject.

In other aspects, the invention may comprise use of a ribozyme. HA4 nucleic acids may be constructed or isolated which, when transcribed, produce RNA enzymes—ribozymes—that can act as endoribonucleases and catalyze the cleavage of RNA molecules with selected sequences. The cleavage of selected messenger RNAs can result in the reduced production of their encoded polypeptide products. These genes may be used to prepare one or more novel cells, tissues and organisms which possess them. The transgenic cells, tissues or organisms may possess reduced levels of polypeptides including, but not limited to, the polypeptides cited above.

B. Hybridization Probes

The nucleic acid segments of the present invention, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. In addition to their use in directing the expression of the HA4 protein, the nucleic acid sequences disclosed herein also have a variety of other uses. For example, they also have utility as probes or primers in nucleic acid hybridization embodiments. As such, it is contemplated that nucleic acid segments that comprise a sequence region that consists of at least a 14 nucleotide-long contiguous sequence that has the same sequence as, or is complementary to, a 14 nucleotide-long contiguous sequence of SEQ ID NO:1, will find particular utility. Longer contiguous identical or complementary sequences will also be of use in certain embodiments.

It will be readily understood that “intermediate lengths,” in this context, means any length between the quoted ranges, such as 14, 15, 16, 17, 18, 19, 20, etc.; 21, 22, 23, etc.; 30, 31, 32, etc.; 50, 51, 52, 53, etc.; 100, 101, 102, 103, etc.; 150, 151, 152, 153, etc.; including all integers through the 200-500; 500-1,000; 1,000-2,000; 2,000-3,000; 3,000-5,000; 5,000-10,000 ranges, up to and including sequences of about 12,001, 12,002, 13,001, 13,002 and the like.

The ability of such nucleic acid probes to specifically hybridize to HA4 encoding sequences will enable them to be of use in detecting the presence of complementary sequences in a given sample. However, other uses are envisioned, including the use of the sequence information for the preparation of mutant species primers, or primers for use in preparing other genetic constructions.

Nucleic acid molecules having sequence regions consisting of contiguous nucleotide stretches of 10, 20, 30, 40, 50, or even of 100-200 nucleotides or so, identical or complementary to SEQ ID NO:1, are particularly contemplated as hybridization probes for use in, e.g., Southern and northern blotting. The inventors have also identified the sequence of genomic DNA for human HA4. The total size of the fragment, as well as the size of the complementary stretch(es), will ultimately depend on the intended use or application of the particular nucleic acid segment. Smaller fragments will generally find use in hybridization embodiments, wherein the length of the contiguous complementary region may be varied, such as between about 10 and about 100 nucleotides, but larger contiguous complementary stretches may be used.

The use of a hybridization probe of about 10-14 nucleotides in length allows the formation of a duplex molecule that is both stable and selective. Molecules having contiguous complementary sequences over stretches greater than 10 bases in length are generally preferred, though, in order to increase stability and selectivity of the hybrid, and thereby improve the quality and degree of specific hybrid molecules obtained, one will generally prefer to design nucleic acid molecules having gene-complementary stretches of 15 to 20 contiguous nucleotides, or even longer where desired.

Hybridization probes may be selected from any portion of any of the sequences disclosed herein. All that is required is to review the sequence set forth in SEQ ID NO:1 and to select any continuous portion of the sequence, from about 10 nucleotides in length up to and including the full length sequence, that one wishes to utilize as a probe or primer. The choice of probe and primer sequences may be governed by various factors, such as, by way of example only, one may wish to employ primers from towards the termini of the total sequence, or from the ends of the functional domain-encoding sequences, in order to amplify further DNA; one may employ probes corresponding to the entire DNA, or to the zinc finger region, or to the proline-rich sequence to clone HA4-type genes from other species or to clone further HA4-like or homologous genes from any species including human; and one may employ wild-type and mutant probes or primers with sequences centered around the zinc finger or proline-rich sequence to screen DNA samples for HA4. Moreover, one may employ probes or primers with sequences centered around the different HA4 isoforms.

The process of selecting and preparing a nucleic acid segment that includes a contiguous sequence from within SEQ ID NO:1 may alternatively be described as preparing a nucleic acid fragment. Of course, fragments may also be obtained by other techniques such as, e.g., by mechanical shearing or by restriction enzyme digestion. Small nucleic acid segments or fragments may be readily prepared by, for example, directly synthesizing the fragment by chemical means, as is commonly practiced using an automated oligonucleotide synthesizer. Also, fragments may be obtained by application of nucleic acid reproduction technology, such as the PCR™ technology of U.S. Pat. No. 4,683,202 and U.S. Pat. No. 4,682,195 (each incorporated herein by reference), by introducing selected sequences into recombinant vectors for recombinant production, and by other recombinant DNA techniques generally known to those of skill in the art of molecular biology.

Accordingly, the nucleotide sequences of the invention may be used for their ability to selectively form duplex molecules with complementary stretches of HA4 genes or cDNAs. Depending on the application envisioned, one will desire to employ varying conditions of hybridization to achieve varying degrees of selectivity of probe towards target sequence. For applications requiring high selectivity, one will typically desire to employ stringent conditions to form the hybrids, e.g., 0.02M-0.15M NaCl at temperatures of 50° C. to 70° C. Such selective conditions tolerate little, if any, mismatch between the probe and the template or target strand, and would be particularly suitable for isolating HA4 genes.

Of course, for some applications, for example, where one desires to prepare mutants employing a mutant primer strand hybridized to an underlying template or where one seeks to isolate HA4 encoding sequences from related species, functional equivalents, or the like, less stringent hybridization conditions will typically be needed in order to allow formation of the heteroduplex. In these circumstances, one may desire to employ conditions such as 0.15M-1.0M salt, at temperatures ranging from 20° C. to 55° C. Cross-hybridizing species can thereby be readily identified as positively hybridizing signals with respect to control hybridizations. In any case, it is generally appreciated that conditions can be rendered more stringent by decreasing NaCl concentrations or by the addition of increasing amounts of formamide, which serves to destabilize the hybrid duplex in the same manner as increased temperature. Thus, hybridization conditions can be readily manipulated, and thus will generally be a method of choice depending on the desired results.

In certain embodiments, it will be advantageous to employ nucleic acid sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of giving a detectable signal. In preferred embodiments, one will likely desire to employ a fluorescent label or an enzyme tag, such as urease, alkaline phosphatase or peroxidase, instead of radioactive or other environmental undesirable reagents. In the case of enzyme tags, calorimetric indicator substrates are known that can be employed to provide a means visible to the human eye or spectrophotometrically, to identify specific hybridization with complementary nucleic acid-containing samples.

In general, it is envisioned that the hybridization probes described herein will be useful both as reagents in solution hybridization as well as in embodiments employing a solid phase. In embodiments involving a solid phase, the test DNA (or RNA) is adsorbed or otherwise affixed to a selected matrix or surface. This fixed, single-stranded nucleic acid is then subjected to specific hybridization with selected probes under desired conditions. The selected conditions will depend on the particular circumstances based on the particular criteria required (depending, for example, on the G+C contents, type of target nucleic acid, source of nucleic acid, size of hybridization probe, etc.). Following washing of the hybridized surface so as to remove nonspecifically bound probe molecules, specific hybridization is detected, or even quantified, by means of the label.

It will also be understood that this invention is not limited to the particular nucleic acid and amino acid sequences of SEQ ID NOS:1 and 2. Recombinant vectors and isolated DNA segments may therefore variously include the HA4 coding regions themselves, coding regions bearing selected alterations or modifications in the basic coding region, or they may encode larger polypeptides that nevertheless include HA4 coding regions or may encode biologically functional equivalent proteins or polypeptides that have variant amino acids sequences.

The DNA segments of the present invention encompass biologically functional equivalent HA4 proteins and polypeptides. Such sequences may arise as a consequence of codon redundancy and functional equivalency that are known to occur naturally within nucleic acid sequences and the proteins thus encoded. Alternatively, functionally equivalent proteins or polypeptides may be created via the application of recombinant DNA technology, in which changes in the protein structure may be engineered, based on considerations of the properties of the amino acids being exchanged. Changes designed by man may be introduced through the application of site-directed mutagenesis techniques, e.g., to introduce improvements to the antigenicity of the protein or to test HA4 mutants in order to examine transcriptional activity at the molecular level.

If desired, one may also prepare fusion proteins and polypeptides, e.g., where the HA4 coding regions are aligned within the same expression unit with other proteins or polypeptides having desired functions, such as for purification or immunodetection purposes (e.g., proteins that may be purified by affinity chromatography or identified by enzyme label coding regions, respectively).

C. Recombinant Vectors and Protein Expression

Recombinant vectors form important further aspects of the present invention. Particularly useful vectors are contemplated to be those vectors in which the coding portion of the DNA segment, whether encoding a full length protein or smaller polypeptide, is positioned under the control of a promoter. The promoter may be in the form of the promoter that is naturally associated with an HA4 gene, e.g., in osteoblasts as may be obtained by isolating the 5′ non-coding sequences' located upstream of the coding segment or exon, for example, using recombinant cloning and/or PCR™ technology, in connection with the compositions disclosed herein (PCR™ technology is disclosed in U.S. Pat. No. 4,683,202 and U.S. Pat. No. 4,682,195, each incorporated herein by reference). Alternatively, the promoter may be a “heterologous” source, i.e., not the native HA4 promoter.

1. Promoters and Enhancers

The promoters and enhancers that control the transcription of protein encoding genes in mammalian cells are composed of multiple genetic elements. The cellular machinery is able to gather and integrate the regulatory information conveyed by each element, allowing different genes to evolve distinct, often complex patterns of transcriptional regulation. Tables 3 and 4 describe suitable promoter/enhancer elements.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator proteins. At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between elements is flexible, so that promoter function is preserved when elements are inverted or moved relative to one another. In the tk promoter, the spacing between elements can be increased to 50 bp apart before activity begins to decline. Depending on the promoter, it appears that individual elements can function either cooperatively or independently to activate transcription.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

TABLE 3

| PROMOTERS | REFERENCES |
|---|---|
| Immunoglobulin | Gilles et al., 1983; Grosschedl and |
| Heavy Chain | Baltimore, 1985; Atchinson and Perry, |
| | 1986, 1987; Imler et al., 1987; |
| | Weinberger et al., 1988; Kiledjian |
| | et al., 1988; Porton et al., 1990 |
| Immunoglobulin | Queen and Baltimore, 1983; Picard |
| Light Chain | and Schaffner, 1984 |
| T-Cell Receptor | Luria et al., 1987, Winoto and |
| | Baltimore, 1989; Redondo et al., |
| | 1990 |
| HLA DQ a and DQ β | Sullivan and Peterlin, 1987 |
| β-Interferon | Goodbourn et al., 1986; Fujita et |
| | al., 1987; Goodbourn and Maniatis, |
| | 1985 |
| Interleukin-2 | Greene et al., 1989 |
| Interleukin-2 Receptor | Greene et al., 1989; Lin et al., |
| | 1990 |
| MHC Class II 5 | Koch et al., 1989 |
| MHC Class II HLA-Dra | Sherman et al., 1989 |
| β-Actin | Kawamoto et al., 1988; Ng et al., |
| | 1989 |
| Muscle Creatine | Jaynes et al., 1988; Horlick and |
| Kinase | Benfield, 1989; Johnson et al., |
| | 1989a |
| Prealbumin | Costa et al., 1988 |
| (Transthyretin) | |
| Elastase I | Omitz et al., 1987 |
| Metallothionein | Karin et al., 1987; Culotta and |
| | Hamer, 1989 |
| Collagenase | Pinkert et al., 1987; Angel et |
| | al., 1987 |
| Albumin Gene | Pinkert et al., 1987, Tronche et |
| | al., 1989, 1990 |
| a-Fetoprotein | Godbout et al., 1988; Campere and |
| | Tilghman, 1989 |
| t-Globin | Bodine and Ley, 1987; Perez-Stable |
| | and Constantini, 1990 |
| β-Globin | Trudel and Constantini, 1987 |
| e-fos | Cohen et al., 1987 |
| c-HA-ras | Triesman, 1986; Deschamps et al., |
| | 1985 |
| Insulin | Edlund et al., 1985 |
| Neural Cell Adhesion | Hirsch et al., 1990 |
| Molecule (NCAM) | |
| a₁-Antitrypain | Latimer et al., 1990 |
| H2B (TH2B) Histone | Hwang et al., 1990 |
| Mouse or Type I | Ripe et al., 1989 |
| Collagen | |
| Glucose-Regulated | Chang et al., 1989 |
| Proteins (GRP94 | |
| and GRP78) | |
| Rat Growth Hormone | Larsen et al., 1986 |
| Human Serum Amyloid | Edbrooke et al., 1989 |
| A (SAA) | |
| Troponin I (TN I) | Yutzey et al., 1989 |
| Platelet-Derived | Pech et al., 1989 |
| Growth Factor | |
| Duchenne Muscular | Klamut et al., 1990 |
| Dystrophy | |
| SV40 | Banerji et al., 1981; Moreau et |
| | al., 1981; Sleigh and Lockett, |
| | 1985; Firak and Subramanian, 1986; |
| | Herr and Clarke, 1986; Imbra and |
| | Karin, 1986; Kadesch and Berg, 1986; |
| | Wang and Calame, 1986; Ondek et al., |
| | 1987; Kuhl et al., 1987 Schaffner et |
| | al., 1988 |
| Polyoma | Swartzendruber and Lehman, 1975; |
| | Vasseur et al., 1980; Katinka et al., |
| | 1980, 1981; Tyndell et al., 1981; |
| | Dandolo et al., 1983; deVilliers et |
| | al., 1984; Hen et al., 1986; Satake |
| | et al., 1988; Campbell and Villarreal, |
| | 1988 |
| Retroviruses | Kriegler and Botchan, 1982, 1983; |
| | Levinson et al., 1982; Kriegler et |
| | al., 1983, 1984a, b, 1988; Bosze et |
| | al., 1986; Miksicek et al., 1986; |
| | Celander and Haseltine, 1987; Thiesen |
| | et al., 1988; Celander et al., 1988; |
| | Chol et al., 1988; Reisman and Rotter, |
| | 1989 |
| Papilloma Virus | Campo et al., 1983; Lusky et al., |
| | 1983; Spandidos and Wilkie, 1983; |
| | Spalholz et al., 1985; Lusky and |
| | Botchan, 1986; Cripe et al., 1987; |
| | Gloss et al., 1987; Hirochika et |
| | al., 1987, Stephens and Hentschel, |
| | 1987; Glue et al., 1988 |
| Hepatitis B Virus | Bulla and Siddiqui, 1986; Jameel |
| | and Siddiqui, 1986; Shaul and |
| | Ben-Levy, 1987; Spandau and Lee, |
| | 1988; Vannice and Levinson, 1988 |
| Human | Muesing et al., 1987; Hauber and |
| Immunodeficiency | Cullan, 1988; Jakobovits et al., |
| Virus | 1988; Feng and Holland, 1988; |
| | Takebe et al., 1988; Rowen et al., |
| | 1988; Berkhout et al., 1989; Laspia |
| | et al., 1989; Sharp and Marciniak, |
| | 1989; Braddock et al., 1989 |
| Cytomegalovirus | Weber et al., 1984; Boshart et al., |
| | 1985; Foecking and Hofstetter, 1986 |
| Gibbon Ape | Holbrook et al., 1987; Quinn et al., |
| Leukemia Virus | 1989 |

| | INDUCER | REFERENCES |
|---|---|---|
| MT II | Phorbol Ester (TFA) | Palmiter et al., 1982; |
| | Heavy metals | Haslinger and Karin, |
| | | 1985; Searle et al., |
| | | 1985; Stuart et al., |
| | | 1985; Imagawa et al., |
| | | 1987; Karin, 1987; |
| | | Angel et al., 1987b; |
| | | McNeall et al., 1989 |
| MMTV (mouse mammary | Glucocorticoids | Huang et al., 1981; |
| tumor virus) | | Lee et al., 1981; |
| | | Majors and Varmus, |
| | | 1983; Chandler et al., |
| | | 1983; Lee et al., |
| | | 1984; Fonta et al., |
| | | 1985; Sakai et al., |
| | | 1986 |
| β-Interferon | Poly(rI)X Poly(rc) | Tavernier et al., |
| | | 1983 |
| Adenovirus 5 E2 | Ela | Imperiale and Nevins, |
| | | 1984 |
| Collagenase | Phorbol Ester (TPA) | Angel et al., 1987a |
| Stromelysin | Phorbol Ester (TPA) | Angel et al., 1987b |
| SV40 | Phorbol Ester (TFA) | Angel et al., 1987b |
| Murine MX Gene | Interferon, | |
| | Newcastle Disease | |
| | Virus | |
| GRP78 Gene | A23187 | Resendez et al., 1988 |
| a-2-Macroglobulin | IL-6 | Kunz et al., 1989 |
| Vimentin | Serum | Rittling et al., 1989 |
| MHC Class I Gene | Interferon | Blanar et al., 1989 |
| H-2kb | | |
| HSP70 | Ela, SV40 Large | Taylor et al., 1989; |
| | T Antigen | Taylor and Kingston, |
| | | 1990a, b |
| Proliferin | Phorbol Ester-TPA | Mordacq and Linzer, |
| | | 1989 |
| Tumor Necrosis | FMA | Hensel et al., 1989 |
| Factor | | |
| Thyroid Stimulating | Thyroid Hormone | Chatterjee et al., |
| Hormone a Gene | | 1989 |

It is understood in the art that to bring a coding sequence under the control of a promoter, one positions the 5′ end of the transcription initiation site of the transcriptional reading frame of the protein between about 1 and about 50 nucleotides “downstream” of (i.e., 3′ of) the chosen promoter. In addition, where eukaryotic expression is contemplated, one will also typically desire to incorporate into the transcriptional unit which includes the cotransporter protein, an appropriate polyadenylation site (e.g., 5′-AATAAA-3′) if one was not contained within the original cloned segment. Typically, the poly-A addition site is placed about 30 to 2000 nucleotides “downstream” of the termination site of the protein at a position prior to transcription termination.

2. Expression Vectors

As mentioned above, in connection with expression embodiments to prepare recombinant HA4 proteins and polypeptides, it is contemplated that longer DNA segments will most often be used, with DNA segments encoding the entire HA4 protein being most preferred. However, it will be appreciated that the use of shorter DNA segments to direct the expression of HA4 polypeptides or epitopic core regions, such as may be used to generate anti-HA4 antibodies, also falls within the scope of the invention.

Once a suitable (full length if desired) clone or clones have been obtained, whether they be cDNA based or genomic, one may proceed to prepare an expression system for the recombinant preparation of HA4. The engineering of DNA segment(s) for expression in a prokaryotic or eukaryotic system may be performed by techniques generally known to those of skill in recombinant expression. It is believed that virtually any expression system may be employed in the expression of HA4.

It is proposed that transformation of host cells with DNA segments encoding the HA4 protein will provide a convenient means for obtaining active HA4. However, separate expression followed by reconstitution is also certainly within the scope of the invention.

Both cDNA and genomic sequences are suitable for eukaryotic expression, as the host cell will generally process the genomic transcripts to yield functional mRNA for translation into protein. Generally speaking, it may be more convenient to employ as the recombinant gene a cDNA version of the gene. It is believed that the use of a cDNA version will provide advantages in that the size of the gene will generally be much smaller and more readily employed to transfect the targeted cell than will a genomic gene, which will typically be up to an order of magnitude larger than the cDNA gene. However, the inventors do not exclude the possibility of employing a genomic version of a particular gene where desired.

In addition, it is possible to express partial sequences, e.g., for the generation of antibodies against discrete portions of a gene product, even when the entire sequence of that gene product remains unknown. As noted herein, computer programs are available to aid in the selection of regions which have potential immunologic significance. For example, software capable of carrying out this analysis is readily available commercially, for example MacVector (IBI, New Haven, Conn.). The software typically uses standard algorithms such as the Kyte/Doolittle or Hopp/Woods methods for locating hydrophilic sequences which are characteristically found on the surface of proteins and are, therefore, likely to act as antigenic determinants.

In the recombinant production of large amounts of proteins or polypeptides, it may be advisable to analyze the protein to detect putative transmembrane sequences. Such sequences are typically very hydrophobic and are readily detected by the use of standard sequence analysis software, such as MacVector (IBI, New Haven, Conn.). The presence of transmembrane sequences is often deleterious when a recombinant protein is synthesized in many expression systems, especially E. coli, as it leads to the production of insoluble aggregates that are difficult to renature into the native conformation of the protein. Deletion of transmembrane sequences typically does not significantly alter the conformation of the remaining protein structure.

Moreover, transmembrane sequences, being by definition embedded within a membrane, are inaccessible. Antibodies to these sequences will not, therefore, generally prove useful in in vivo or in situ studies. Deletion of transmembrane-encoding sequences from the genes used for expression can be achieved by standard techniques. For example, fortuitously-placed restriction enzyme sites can be used to excise the desired gene fragment, or PCR™-type amplification can be used to amplify only the desired part of the gene.

As used herein, the terms “engineered” and “recombinant” cells are intended to refer to a cell into which an exogenous DNA segment or gene, such as a cDNA or gene encoding an HA4 protein or polypeptide has been introduced. Therefore, engineered cells are distinguishable from naturally occurring cells which do not contain a recombinantly introduced exogenous DNA segment or gene. Engineered cells are thus cells having a gene or genes introduced through the hand of man. Recombinant cells include those having an introduced cDNA or genomic gene, and also include genes positioned adjacent to a promoter not naturally associated with the particular introduced gene.

To express a recombinant HA4 protein or polypeptide, whether mutant or wild-type, in accordance with the present invention one would prepare an expression vector that comprises an HA4 protein or polypeptide-encoding nucleic acid segment under the control of one or more promoters. To bring a coding sequence “under the control of” a promoter, one positions the 5′ end of the transcription initiation site of the transcriptional reading frame generally between about 1 and about 50 nucleotides “downstream” of (i.e., 3′ of) the chosen promoter. The “upstream” promoter stimulates transcription of the DNA and promotes expression of the encoded recombinant protein. This is the meaning of “recombinant expression” in this context.

i) Host Cells

Many standard techniques are available to construct expression vectors containing the appropriate nucleic acids and transcriptional/translational control sequences in order to achieve protein or polypeptide expression in a variety of host-expression systems. Cell types available for expression include, but are not limited to, bacteria, such as E. coli and B. subtilis transformed with recombinant bacteriophage DNA, plasmid DNA or cosmid DNA expression vectors.

Certain examples of prokaryotic hosts are E. coli strain RR1, E. coli LE392, E. coli B, E. coli X 1776 (ATCC No. 31537) as well as E. coli W3110 (F—, lambda-, prototrophic, ATCC No. 273325); bacilli such as Bacillus subtilis; and other enterobacteriaceae such as Salmonella typhimurium, Serratia marcescens, and various Pseudomonas species.

In general, plasmid vectors containing replicon and control sequences which are derived from species compatible with the host cell are used in connection with these hosts. The vector ordinarily carries a replication origin, as well as marking sequences which are capable of providing phenotypic selection in transformed cells. For example, E. coli is often transformed using pBR322, a plasmid derived from an E. coli species. pBR322 contains genes for ampicillin and tetracycline resistance and thus provides means for identifying transformed cells. The pBR plasmid, or other microbial plasmid or phage must also contain, or be modified to contain, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as E. coli LE392.

Further useful vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, mannose binding protein (MBP) and the like.

Promoters that are most commonly used in recombinant DNA construction include the β-lactamase (penicillinase), lactose and tryptophan (trp) promoter systems. While these are the most commonly used, other microbial promoters have been discovered and utilized, and details concerning their nucleotide sequences have been published, enabling those of skill in the art to ligate them functionally with plasmid vectors.

The following details concerning recombinant protein production in bacterial cells, such as E. coli, are obtained from exemplary information on recombinant protein production in general, the adaptation of which to a particular recombinant expression system will be known to those of skill in the art.

Bacterial cells, for example, E. coli, containing the expression vector are grown in any of a number of suitable media, for example, LB. The expression of the recombinant protein may be induced, e.g., by adding IPTG to the media or by switching incubation to a higher temperature. After culturing the bacteria for a further period, generally of between 2 and 24 hours, the cells are collected by centrifugation and washed to remove residual media.

The bacterial cells are then lysed, for example, by disruption in a cell homogenizer and centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed.

If the recombinant protein is expressed in the inclusion bodies, as is the case in many instances, these can be washed in any of several solutions to remove some of the contaminating host proteins, then solubilized in solutions containing high concentrations of urea (e.g., 8M) or chaotropic agents such as guanidine hydrochloride in the presence of reducing agents, such as β-mercaptoethanol or DTT (dithiothreitol).

Under some circumstances, it may be advantageous to incubate the protein for several hours under conditions suitable for the protein to undergo a refolding process into a conformation which more closely resembles that of the native protein. Such conditions generally include low protein concentrations, less than 500 μg/ml, low levels of reducing agent, concentrations of urea less than 2 M and often the presence of reagents such as a mixture of reduced and oxidized glutathione which facilitate the interchange of disulfide bonds within the protein molecule.

The refolding process can be monitored, for example, by SDS-PAGE, or with antibodies specific for the native molecule (which can be obtained from animals immunized with the native molecule or smaller quantities of recombinant protein). Following refolding, the protein can then be purified further and separated from the refolding mixture by chromatography on any of several supports including ion exchange resins, gel permeation resins or on a variety of affinity columns.

In addition to prokaryotes, eukaryotic microbes, such as yeast cultures, may also be used. Saccharomyces cerevisiae, or common baker's yeast, is the most commonly used among eukaryotic microorganisms, although a number of other strains are commonly available. For expression in Saccharomyces, the plasmid YRp7, for example, is commonly used (Stinchcomb et al., 1979; Kingsman et al., 1979; Tschemper et al., 1980). This plasmid already contains the trp1 gene which provides a selection marker for a mutant strain of yeast lacking the ability to grow in tryptophan, for example ATCC No. 44076 or PEP4-1 (Jones, 1977). The presence of the trp1 lesion as a characteristic of the yeast host cell genome then provides an effective environment for detecting transformation by growth in the absence of tryptophan.

Suitable promoting sequences in yeast vectors include the promoters for 3-phosphoglycerate kinase (Hitzeman et al., 1980) or other glycolytic enzymes (Hess et al., 1968; Holland et al., 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other suitable promoters, which have the additional advantage of transcription controlled by growth conditions, include the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization.

In addition to micro-organisms, cultures of cells derived from multicellular organisms may also be used as hosts. In principle, any such cell culture is workable, whether from vertebrate or invertebrate culture. In addition to mammalian cells, these include insect cell systems infected with recombinant virus expression vectors (e.g., baculovirus); and plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing one or more HA4 protein or polypeptide coding sequences.

In a useful insect system, Autographica californica nuclear polyhidrosis virus (AcNPV) is used as a vector to express foreign genes. The virus grows in Spodoptera frugiperda cells. The HA4 protein or polypeptide coding sequences are cloned into non-essential regions (for example the polyhedrin gene) of the virus and placed under control of an AcNPV promoter (for example the polyhedrin promoter). Successful insertion of the coding sequences results in the inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (e.g., U.S. Pat. No. 4,215,051).

Examples of useful mammalian host cell lines are VERO and HeLa cells, Chinese hamster ovary (CHO) cell lines, W138, BHK, COS-7, 293, HepG2, 3T3, RIN and MDCK cell lines. In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein.

Different host cells have characteristic and specific mechanisms for the post-translational processing and modification of proteins. Appropriate cell lines or host systems can be chosen to ensure the correct modification and processing of the foreign protein expressed. To this end, eukaryotic host cells which possess the cellular machinery for glycosylation, intracellular transport, high expression and DNA replication may be used if desired, with a cell that allows for high expression being preferred.

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Non-limiting examples of virus vectors that may be used to deliver a nucleic acid of the present invention are described below.

ii) Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

iii) AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

iv) Retroviral Vectors

Retroviruses are valuable delivery vectors in due, in part, to their ability to integrate their genes into the host genome, transferring a large amount of foreign genetic material, infecting a broad spectrum of species and cell types and of being packaged in special cell-lines (Miller, 1992). In order to construct a retroviral vector, a nucleic acid of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

v) Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

vi) Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989): Using antibodies against major histocompatibility complex class I and class II antigens, they demonstrated the infection of a variety of human cells that bore those surface antigens with an ecotropic virus in vitro (oux et al., 1989).

vii) Other Signals

Specific initiation signals may also be required for efficient translation of HA4 coding sequences. These signals include the ATG initiation codon and adjacent Kosak sequences. Exogenous translational control signals, including the ATG initiation codon, may additionally need to be provided. One of ordinary skill in the art would readily be capable of determining this and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators (Bittner et al., 1987).

In eukaryotic expression, one will also typically desire to incorporate into the transcriptional unit an appropriate polyadenylation site (e.g., 5′-AATAAA-3′) if one was not contained within the original cloned segment. Typically, the poly A addition site is placed about 30 to 2000 nucleotides “downstream” of the termination codon of the protein at a position prior to transcription termination.

For long-term, high-yield production of recombinant HA4 proteins, stable expression is preferred. For example, cell lines that stably express constructs encoding HA4 proteins or polypeptides may be engineered. Rather than using expression vectors that contain viral origins of replication, host cells can be transformed with vectors controlled by appropriate expression control elements (e.g., promoter, enhancer, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines.

viii) Selection Systems

A number of selection systems may be used, including, but not limited, to the herpes simplex virus thymidine kinase (Wigler et al., 1977), hypoxanthine-guanine phosphoribosyltransferase (Szybalska et al., 1962) and adenine phosphoribosyltransferase genes (Lowry et al., 1980), in tk-, hgprt- or aprt- cells, respectively. Also, anti-metabolite resistance can be used as the basis of selection for dhfr, that confers resistance to methotrexate (Wigler et al., 1980; O'Hare et al., 1981); gpt, that confers resistance to mycophenolic acid (Mulligan et al., 1981); neo, that confers resistance to the aminoglycoside G418 (Colberre-Garapin et al., 1981); and hygro, that confers resistance to hygromycin (Santerre et al., 1984).

It is contemplated that the HA4 of the invention may be “overexpressed,” i.e., expressed in increased levels relative to its natural expression in osteoblast cells, or even relative to the expression of other proteins in the recombinant host cell. Such overexpression may be assessed by a variety of methods, including radio-labeling and/or protein purification. However, direct methods are preferred, for example, those involving SDS/PAGE and protein staining or western blotting, followed by quantitative analyses, such as densitometric scanning of the resultant gel or blot. A specific increase in the level of the recombinant protein or polypeptide in comparison to the level in natural osteoblasts is indicative of overexpression, as is a relative abundance of the specific protein in relation to the other proteins produced by the host cell and, e.g., visible on a gel.

### IV. DEVELOPMENT OF HA4-RELATED AGENTS AND ASSAYS

It is contemplated that the HA4-related agents described herein will be useful in many areas, for example in screening assays, monitoring amounts and qualities of HA4 in clinical samples or to target the expression of foreign genes into osteoblasts, all as described in more detail herein. As used herein, the term “HA4-related agents” refers to full length as well as partial DNA segments; other members of the HA4 family; isolated and purified native HA4 as well as recombinantly produced HA4; antibodies raised to any of the above forms; cells and animals engineered to overproduce HA4.

The HA4-related agents described herein may, of course, additionally be used to search for molecules that modulate the expression and/or function of HA4 (e.g., naturally occurring proteins, chemicals, synthetic peptides, carbohydrates, lipids, recombinant proteins, cell extracts, and supernatant, etc.). This may, for example, involve the use of HA4 transfectants to search for molecules that bind to HA4 in the cell to enhance its activity thereby enhancing bone production.

Another contemplated use of the agents of the invention is to regulate cell differentiation for example, to regulate the differentiation of precursor cells, such as mesenchymal precursor cells, to form osteoblasts. In another example one may establish osteoblast lines by introducing HA4 promoters. This may be accomplished by using the 5′-flanking region of the HA4 gene to drive cellular differentiation toward osteoblasts or by using oncogenes (e.g., c-myc) driven by osteoblast-specific promoters.

A. HA4-Related Agents and Assays

The following reagents are included in the present invention as “HA4-related reagents”: (a) DNA segments of HA4, including the 5′- and 3′-flanking regions, (b) RNA segments of sense or anti-sense strands of HA4, including truncated or mutated transcripts, (c) HA4 polypeptides or proteins, including truncated or mutated forms and their biological equivalents, (d) polyclonal or monoclonal antibodies against HA4, (e) cell lines that express HA4, (f) vectors designed to produce HA4 polypeptides or proteins, (g) cell lines that are engineered to express HA4, and (h) transgenic animals lacking at least one functional HA4 allele, or comprising an expression cassette with an HA4 promoter linked to a screenable marker.

The following assays that employ HA4-related reagents are also included in the present invention as “HA4-related assays”: (a) assays to detect HA4 DNA, including Southern blotting, genomic PCR™, colony and plaque hybridization, and slot blotting; (b) assays to detect HA4 RNA, including northern blotting, RT-PCR™, in situ hybridization, primer extension assay, and RNase protection assay, (c) assays to detect HA4 polypeptides or proteins, including ELISA, Western blotting, immunoprecipitation, radioimmuno-absorption and -competition assays, and immunofluorescence and immunohistochemical stainings; and (d) assays to search for agents that modulate HA4 expression and/or function. Detailed methodologies for these assays will be described in the following sections.

B. Assays to Examine HA4 Nucleic Acids

Nucleic acid segments of HA4 or related molecules that exhibit significant homologies with, or that contain portions of HA4 will be used as probes to detect members of the HA4 family of genes. The HA4 family of genes is defined as genes that are detectable with at least one of these probes. For this purpose, standard assays, including Southern blotting, PCR™, colony and plaque hybridization, and slot blot hybridization will be employed under various conditions with different degrees of stringency as described previously. Specimens to be tested include cDNA libraries, genomic DNA, cDNA, and DNA fragments isolated from cells or tissues. These assays may be modified to detect selectively mutated HA4 DNA. For this purpose, Southern blotting or PCR™ will be employed to detect or amplify the mutated DNA segments. These segments will then be sequenced to identify the mutated nucleotides. Alternatively, a combination of selected restriction enzymes will be employed to reveal molecular heterogeneity in Southern blotting. Moreover, these assays may be modified to detect selectively different domains or different portions of the HA4 nucleotide sequences. For this aim, one may employ probes or primers for different portions of the nucleotide sequences. More sophisticated methods may be employed to screen point mutations. For example, it is contemplated that one may choose a PCR™-single-strand conformation polymorphism (PCR™-SSCP) analysis (Sarkar et al., 1995).

Nucleotides of HA4 (SEQ ID NO:1) or related nucleotides that exhibit significant homologies with, or that contain portions of HA4 will be used as probes to detect transcripts of the HA4 family of genes. For this purpose, standard assays, including northern blotting, RT-PCR™, in situ hybridization, primer extension assay and RNase protection assay will be employed under various conditions with different degrees of stringency as described previously. Specimens to be tested include total RNA and mRNA isolated from cells or tissues and cell and tissue samples themselves obtained from living animals or patients. These assays may be modified to detect selectively the transcripts for different domains or different isoforms. For this purpose, the inventors will employ probes or primers for different portions of the nucleotide sequences. Northern blotting may be used to detect selectively different isoforms. For this purpose, oligonucleotide probes will be constructed, each covering different portions of the nucleotide sequences. To define the nucleotides that are deleted from the original sequence, RNase protection assays may be employed. Detection of mutated RNA is also included in the present invention. For this aim, RNA isolated from osteoblasts will be analyzed by northern blotting or RT-PCR™.

It is further contemplated that assays may be designed to detect selectively different RNA species. Similar methods using RT-PCR™ may be employed to identify spliced variants and even other isoforms that are produced by other mechanisms. Alternatively, Northern blotting may be used to detect selectively different isoforms. For this purpose, oligonucleotide probes will be constructed, each covering different portions of the nucleotide sequences. To define the nucleotides that are deleted from the original sequence, RNase protection assays may be employed.

C. Assays to Examine HA4 at Protein or Polypeptide Levels

Antibodies against HA4 will be used to detect HA4 proteins or polypeptides. For this purpose, standard assays, including ELISA, western blotting, immunoprecipitation, radioimmuno-absorption and radioimmuno-competition assays, and immunofluorescence and immunohistochemical stainings will be employed under various conditions with different degrees of specificity and sensitivity. Specimens to be tested include viable cells, whole cellular extracts, and different subcellular fractions of established cell lines, as well as cells, tissues, and body fluids isolated from living animals or patients. These assays may be modified to detect selectively different epitopes, domains, or isoforms of HA4 polypeptides or proteins. For this purpose, the inventors will develop and employ a panel of MAb against different epitopes or domains.

D. Assays to Search for Reagents That Modulate the Activity of HA4 and the Expression of HA4 Gene

Finally, the HA4-related assays described above may also be used to search for molecules that modulate HA4-dependent activity, comprising admixing a HA4 expressing cell with a candidate substance and identifying if the candidate substance inhibits/stimulates the expression of HA4. The HA4 expressing cell may be an osteoblast. Alternatively, the HA4 expressing cell may comprise an engineered cell that expresses recombinant HA4.

Screening will determine whether the candidate substance affects the expression of HA4. For this purpose, cells will be treated with the candidate substance(s) either individually or in combination and then examined for enhanced HA4 activity at the levels of mRNA, protein, and function. Alternatively, the candidate substances may be tested in vivo by administering into live animals such as mice. In this case, cells of interest will be isolated from mice after treatment with the candidate substance(s) or combinations thereof and examined in vitro for enhanced HA4 activity, once again, by measuring the levels of mRNA, protein, and/or function. In performing these assays, it will be important to also examine the effect(s) of candidate substances on the activity of different isoforms of HA4. In preferred embodiments, agents that enhance or stimulate HA4 expression will be formulated in a pharmaceutical acceptable medium.

A candidate substance(s) that inhibits the activity of HA4 within osteoblasts may be identified by inhibition of osteoblast differentiation or bone formation. The invention thus, provides agents that inhibit HA4-mediated activation of osteoblasts. In preferred embodiments, the agent of the invention will be formulated in a pharmaceutical acceptable medium.

In further embodiments, the present invention concerns a method for identifying new osteoblast interaction inhibitory/stimulatory compounds, which may be termed as “candidate substances.” It is contemplated that this screening technique will prove useful in the general identification of a compound that will serve the purpose of inhibiting/stimulating osteoblast activation. Stimulators of osteoblast activation have therapeutic applications in diseases such as osteoporosis, bone reconstructions in bone fracture repair etc.

It is further contemplated that useful compounds in this regard will in no way be limited to antibodies. In fact, it may prove to be the case that the most useful pharmacological compounds for identification through application of the screening assay will be non-peptidyl in nature and serve to inhibit the osteoblast activation process through a tight binding or other chemical interaction.

Candidate molecules may be examined for their capacities to suppress or to enhance the expression of HA4 by osteoblasts at mRNA or protein levels. For this aim, osteoblasts will be incubated with test samples and then examined for HA4 expression by northern blotting, RT-PCR™, in situ hybridization, primer extension assay and RNase protection assay (at RNA levels) or by ELISA, western blotting, immunoprecipitation, radioimmuno-absorption and competition assays, and immunofluorescence and immunohistochemical stainings (at protein levels).

While a candidate substance may be any type of substance that may interact with HA4 to enhance its activity and stimulate bone formation, one preferred method for obtaining candidate substances will be by utilizing combinatorial chemistry techniques. Such techniques are well known to the skilled artisan and include methods as described in VanHijfte et al. (1999) and Floyd et al. (1999), both incorporated herein by reference.

E. Transgenic Animals and Cells and HA4 Knockouts

1. Transgenic Animal and Cells

Cells, cell lines and animals deficient for the HA4 gene can be generated and utilized, for example, as part of the identification of specific modulators such as stimulators or inhibitors of osteoblast gene expression and activity in addition to the identification assays described above. Thus, HA4 deficient cells, cell lines and animals will frequently be used herein as a representative example.

The term “HA4-deficient,” as used herein, refers to cells, cell lines and/or animals which exhibit a lower level of functional HA4 activity than corresponding cells, or cell lines or animals whose cells, contain two normal, wild type copies of the HA4 gene. A representative HA4-deficient, or “knockout” animal is a mouse HA4-deficient animal. Knockout animals are well known to those of skill in the art. See, for example, Horinouchi et al. (1995); and Otterbach and Stoffel (1995), both of which are incorporated herein by reference in their entirety. Techniques for generating additional HA4 knockout cells, cell lines and animals are described below. Cells that are heterozygous and homozygous for knock-outs are contemplated.

Cells and cell lines deficient in HA4 activity can be derived from HA4 knockout animals, utilizing standard techniques well known to those of skill in the art. Such animals may be used to derive a cell line which may be used as an assay substrate in culture. While primary cultures may be utilized, the generation of continuous cell lines is preferred. For examples of techniques which may be used to derive a continuous cell line from the transgenic animals, see Small et al., 1985. Such techniques for generating cells and cell lines can also be utilized in the context of the transgenic and genetically engineered animals described below.

With respect to HA4 deficient cells, such cells can, for example, include cells taken from and cell lines derived from patients exhibiting bone disorders, such as osteoporosis. Additional HA4-deficient cells and cell lines can be generated using well known recombinant DNA techniques such as, for example, site-directed mutagenesis, to introduce mutations into HA4 gene sequences which will disrupt HA4 activity.

HA4-deficient cells and animals can be generated using the HA4 nucleotide sequences described in the present invention. Such animals can be any species, including but not limited to mice, rats, rabbits, guinea pigs, pigs, micro-pigs, and non-human primates, e.g. baboons, squirrel monkeys and chimpanzees.

Any technique known in the art may be used to introduce a transgene, such as an inactivating gene sequence, into animals to produce the founder lines of transgenic animals. Such techniques include, but are not limited to pronuclear microinjection (U.S. Pat. No. 4,873,191); retrovirus mediated gene transfer into germ lines (Van der Putten et al., 1985); gene targeting in embryonic stem cells (Thompson et al., 1989); electroporation of embryos (Lo, 1983); and sperm-mediated gene transfer (Lavitrano et al., 1989). For a review of such techniques, see Gordon, 1989, which is incorporated by reference herein in its entirety.

As listed above, standard embryonal stem cell (ES) techniques can, for example, be utilized for generation of HA4 knockouts. ES cells can be obtained from preimplantation embryos cultured in vitro (see, e.g., Evans et al., 1981; Bradley et al., 1984; Gossler et al., 1986; Robertson et al., 1986; Wood et al., 1993) The introduced ES cells thereafter colonize the embryo and contribute to the germ line of a resulting chimeric animal (Jaenisch, 1988).

To accomplish HA4 gene disruptions, the technique of site-directed inactivation via gene targeting may be used (Thomas and Capecchi, 1987; reviewed in Frohman et al., 1989; Cappecchi, 1989; Barribault et al., 1989; Wagner, 1990; and Bradley et al., 1992).

Further, standard techniques such as, for example, homologous recombination, coupled with HA4 sequences, can be utilized to inactivate or alter any HA4 genetic region desired. A number of strategies can be utilized to detect or select rate homologous recombinants. For example, PCR can be used to screen pools of transformant cells for homologous insertion, followed by screening of individual clones (Kim et al., 1988; Kim et al., 1991). Alternatively, a positive genetic selection approach can be taken in which a marker gene is constructed which will only be active if homologous insertion occurs, allowing these recombinants to be selected directly (Sedivy et al., 1989). Additionally, the positive-negative approach (PNS) method can be utilized (Mansour et al., 1988; Capecchi, 1989; Capecchi, 1989). Utilizing the PNS method, nonhomologous recombinants are selected against by using the Herpes Simplex virus thymidine kinase (HSV-TK) gene and selecting against its nonhomologous insertion with herpes drugs such as ganciclovir or FIAU. By such counter-selection, the number of homologous recombinants in the surviving transformants is increased.

ES cells generated via techniques such as these, when introduced into the germline of a nonhuman animal make possible the generation of non-mosaic, i.e., non-chimeric progeny. Such progeny will be referred to herein as founder animals. Once the founder animals are produced, they may be bred, inbred, outbred, or crossbred to produce colonies of the particular animal.

Taking as an example of the above, the generation of a HA4 knockout mouse, first, standard techniques can be utilized to isolate mouse HA4 genomic sequences. Such sequences can be routinely isolated by utilizing standard molecular techniques and human HA4 nucleotide sequences as probes and/or as PCR primers, as discussed below.

An inactive allele of the HA4 gene can then be generated by targeted mutagenesis using standard procedures of combined positive and negative selection for homologous recombination in embryonic stem (ES) cells. HA4 genomic clones can be isolated, for example, from a 129/sv mouse genomic library, which is isogenic with the ES cells to be used for gene targeting. The null targeting vector can be constructed containing homologous sequences flanking both 5′ and 3′ sides of a deletion. The vector carries a resistance marker, e.g., a neomycin resistance marker (Neo) for positive selection and a negative marker, e.g., a thymidine kinase (TK) marker, for negative selection.

Briefly, vector DNA can be electroporated into W9.5 ES cells (male-derived), which can then be cultured and selected on feeder layers of mouse embryonic fibroblasts derived from transgenic mice expressing a Neo gene. G418 (350 mg/ml; for gain of Neo) and ganciclovir (2 mM; for loss of TK) can be added to the culture medium to select for resistant ES cell colonies that have undergone homologous recombination at the URO-D gene. Recombinants are identified by screening genomic DNA from ES cell colonies by Southern blot hybridization analysis. Correctly targeted ES cell clones, which also carry a normal complement of 40 chromosomes, can be used to derive mice carrying the mutation. ES cells can be micro-injected into blastocysts at 3.5 days post-coitum obtained from C57BL/6J mice, and blastocysts will be re-implanted into pseudopregnant female mice, which serve as foster mothers. Chimeric progeny derived largely from the ES cells will be identified by a high proportion of agouti coat color (the color of the 129/sv strain of origin of the ES cells) against the black coat color derived from the C57BL/6J host blastocyst. Male chimeric progeny will be tested for germline transmission of the mutation by breeding with C57BL/6J females. Agouti progeny derived from these crosses will be expected to be heterozygous for the mutation, which will be confirmed by Southern blot analysis. These F1 heterozygous progeny will be inter-bred to generate F2 litters containing progeny of all three genotypes (wild-type, heterozygous and homozygous mutants) for phenotypic analyses.

2. Methods of Making Transgenic Animals

Thus, a particular embodiment of the present invention provides transgenic animals which are knockouts for the HA4 gene and thus serve as models for bone disorders involving HA4 and also provides an assay system for identification of modulators which includes both inhibitors and stimulators of HA4 gene expression as well as HA4 functional activity.

Although the present discussion refers to transgenic mice, it is understood that mice are merely exemplary model animal, and any other mammalian animal routinely used as model animal (e.g., rat, guinea pig, rabbit, cats, dogs, pigs and the like) may be generated using the technology described herein. In a general aspect, a transgenic animal is produced by the integration of a given transgene into the genome in a manner that permits the expression of the transgene. The terms “animal” and “non-human animal,” as used herein, include all vertebrate animals, except humans. It also includes individual animals in all stages of development, including embryonic and fetal stages. A “transgenic animal” is any animal containing one or more cells bearing genetic information received, directly or indirectly, by deliberate genetic manipulation at the subcellular level. The genetic manipulation can be performed by any method of introducing genetic material to a cell, including, but not limited to, microinjection, infection with a recombinant virus, particle bombardment or electroporation. The term is not intended to encompass classical cross-breeding or in vitro fertilization, but rather is meant to encompass animals in which one or more cells receive a recombinant DNA molecule. This molecule may be integrated within a chromosome, or it may be extrachromosomally replicating DNA. The genetic information may be foreign to the species of animal to which the recipient belongs, foreign only to the individual recipient, or genetic information already possessed by the recipient expressed at a different level, a different time, or in a different location than the native gene.

Methods for producing transgenic animals are generally described by Wagner and Hoppe (U.S. Pat. No. 4,873,191; which is incorporated herein by reference), Brinster et al. (1985); which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety).

Typically, a gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. Within a particularly preferred embodiment, transgenic mice are generated which are knockouts of HA4.

DNA clones for microinjection can be prepared by any means known in the art. For example, DNA clones for microinjection can be cleaved with enzymes appropriate for removing the bacterial plasmid sequences, and the DNA fragments electrophoresed on 1% agarose gels in TBE buffer, using standard techniques. The DNA bands are visualized by staining with ethidium bromide, and the band containing the expression sequences is excised. The excised band is then placed in dialysis bags containing 0.3 M sodium acetate, pH 7.0. DNA is electroeluted into the dialysis bags, extracted with a 1:1 phenol:chloroform solution and precipitated by two volumes of ethanol. The DNA is redissolved in 1 ml of low salt buffer (0.2 M NaCl, 20 mM Tris, pH 7.4, and 1 mM EDTA) and purified on an Elutip-D™ column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml high salt buffer and precipitated by two volumes of ethanol. DNA concentrations are measured by absorption at 260 nm in a UV spectrophotometer. For microinjection, DNA concentrations are adjusted to 3 μg/ml in 5 mM Tris, pH 7.4 and 0.1 mM EDTA.

Other methods for purification of DNA for microinjection are described in Hogan et al. (1986), in Palmiter et al. (1982); in The Qiagenologist, Application Protocols, 3rd edition, published by Qiagen, Inc., Chatsworth, Calif.; and in Sambrook et al. (2001).

Female mice are induced to superovulate, e.g., by using an injection of pregnant mare serum gonadotropin (PMSG; Sigma) followed, 48 hours later, by an injection of human chorionic gonadotropin (hCG; Sigma). Females are placed with males immediately after hCG injection. Twenty-one hours after hCG injection, the mated females are sacrificed by CO₂ asphyxiation or cervical dislocation and embryos are recovered from excised oviducts and placed in Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with a humidified atmosphere at 5% CO₂, 95% air until the time of injection. Embryos can be implanted at the two-cell stage.

Twenty-five μg of a SalI-linearized SGC targeting vector is electroporated into 1×10⁷ embryonic stem (ES) cells. After a suitable period of incubation, e.g. 36 hr, the transfected cells are then selected using G418 and FIAU. The G418-FIAU-resistant ES colonies are picked into 96-well plates (Ramirez-Solis et al., 1993). Positive ES clones are injected into C57BL/6 blastocysts and transferred into pseudopregnant ICR female recipients. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos transferred. After the transfer, the incision is closed by two sutures.

The resulting male chimeras are bred with C57BL/6 females. Germline transmission can be screened by using a phenotype, such as coat color and confirmed by Southern analysis.

As noted above, transgenic animals and cell lines derived from such animals may find use in certain testing experiments. In this regard, HA4 transgenic animals and cell lines may be exposed to test substances. These test substances can be screened for the ability to induce differentiation of cells to osteoblasts. Compounds identified by such procedures will be useful in the treatment of bone disorders such as osteoporosis. Thus the compounds identified may be used to prevent, treat, ameliorate bone loss.

(i) ES Cells

ES cells are obtained from pre-implantation embryos cultured in vitro (Evans et al., 1981; Bradley et al., 1984; Gossler et al., 1986; Robertson et al., 1986). Transgenes are introduced into ES cells using a number of means well known to those of skill in the art. The transformed ES cells can thereafter be combined with blastocysts from a non-human animal. The ES cells thereafter colonize the embryo and contribute to the germ line of the resulting chimeric animal (for a review see Jaenisch, 1988).

Once the DNA is introduced, e.g., by electroporation (Quillet et al., 1988; Machy et al., 1988), the cells are cultured under conventional conditions well known in the art. In order to facilitate the recovery of those cells which have received the DNA molecule containing the desired gene sequence, it is preferable to introduce the DNA containing the desired gene sequence in combination with a second gene sequence which would contain a detectable marker gene sequence. For the purposes of the present invention, any gene sequence whose presence in a cell permits one to recognize and clonally isolate the cell may be employed as a detectable (selectable) marker gene sequence. The presence of the detectable (selectable) marker sequence in a recipient cell may be recognized by PCR, by detection of radiolabeled nucleotides, or by other assays of detection which do not require the expression of the detectable marker sequence. Typically, the detectable marker gene sequence will be expressed in the recipient cell, and will result in a selectable phenotype. Selectable markers are well known to those of skill in the art. Some examples include the hprt gene, the neo gene, the tk (thyroidinc kinase) gene of herpes simplex virus (Giphart-Gassler et al., 1989), or other genes which confer resistance to amino acid or nucleoside analogues, or antibiotics, etc.

Any ES cell may be used in accordance with the present invention. It is, however, preferred to use primary isolates of ES cells. Such isolates may be obtained directly from embryos such as the CCE cell line, or from the clonal isolation of ES cells from the CCE cell line (Schwartzberg et al., 1989). The purpose of such clonal propagation is to obtain ES cells which have a greater efficiency for differentiating into an animal. Clonally selected ES cells are approximately 10-fold more effective in producing transgenic animals than the progenitor cell line CCE.

(ii) Homologous recombination

Homologous recombination (Koller and Smithies, 1992), directs the insertion of the transgene to a specific location. This technique allows the precise modification of existing genes, and overcomes the problems of positional effects and insertional inactivation observed with transgenic animals generated by pronuclear injection or use of viral vectors. Additionally, it allows the inactivation of specific genes as well as the replacement of one gene for another. In particular embodiments, the DNA segment comprises two selected DNA regions that flank the HA4 coding region, thereby directing the homologous recombination of the coding region into the genomic DNA of a non-human animal species.

Thus, a preferred method for the delivery of transgenic constructs involves the use of homologous recombination. Homologous recombination relies, like antisense, on the tendency of nucleic acids to base pair with complementary sequences. In this instance, the base pairing serves to facilitate the interaction of two separate nucleic acid molecules so that strand breakage and repair can take place. In other words, the “homologous” aspect of the method relies on sequence homology to bring two complementary sequences into close proximity, while the “recombination” aspect provides for one complementary sequence to replace the other by virtue of the breaking of certain bonds and the formation of others.

Put into practice, homologous recombination is used as follows. First, the target gene is selected within the host cell. Sequences homologous to the target gene are then included in a genetic construct, along with some mutation that will render the target gene inactive (stop codon, interruption, and the like). The homologous sequences flanking the inactivating mutation are said to “flank” the mutation. Flanking, in this context, simply means that target homologous sequences are located both upstream (5′) and downstream (3′) of the mutation. These sequences should correspond to some sequences upstream and downstream of the target gene. The construct is then introduced into the cell, thus permitting recombination between the cellular sequences and the construct.

As a practical matter, the genetic construct will normally act as far more than a vehicle to interrupt the gene. For example, it is important to be able to select for recombinants and, therefore, it is common to include within the construct a selectable marker gene. This gene permits selection of cells that have integrated the construct into their genomic DNA by conferring resistance to various biostatic and biocidal drugs. In addition, a heterologous gene that is to be expressed in the cell also may advantageously be included within the construct. The arrangement might be as follows:

| | | | |
|---|---|---|---|
| . . . vector-5′-flanking | sequence·heterologous | gene· | selectable marker |

gene·flanking sequence-3′·vector . . .

Thus, using this kind of construct, it is possible, in a single recombinatorial event, to (i) “knock out” an endogenous gene, (ii) provide a selectable marker for identifying such an event and (iii) introduce a transgene for expression.

Another refinement of the homologous recombination approach involves the use of a “negative” selectable marker. This marker, unlike the selectable marker, causes death of cells which express the marker. Thus, it is used to identify undesirable recombination events. When seeking to select homologous recombinants using a selectable marker, it is difficult in the initial screening step to identify proper homologous recombinants from recombinants generated from random, non-sequence specific events. These recombinants also may contain the selectable marker gene and may express the heterologous protein of interest, but will, in all likelihood, not have the desired “knock out” phenotype. By attaching a negative selectable marker to the construct, but outside of the flanking regions, one can select against many random recombination events that will incorporate the negative selectable marker. Homologous recombination should not introduce the negative selectable marker, as it is outside of the flanking sequences. Examples of processes that use negative selection to enrich for homologous recombination include the disruption of targeted genes in embryonic stem cells or transformed cell lines (Mortensen, 1993; Willnow and Herz, 1994) and the production of recombinant virus such as adenovirus (Imler et al., 1995).

Since the frequency of gene targeting is heavily influenced by the origin of the DNA being used for targeting, it is beneficial to obtain DNA that is as similar (isogenic) to the cells being targeted as possible. One way to accomplish this is by isolation of the region of interest from genomic DNA from a single colony by long range PCR. Using long range PCR it is possible to isolate fragments of 7-12 kb from small amounts of starting DNA.

Gene trapping is a useful technique suitable for use with the present invention. This refers to the utilization of the endogenous regulatory regions present in the chromosomal DNA to activate the incoming transgene. In this way expression of the transgene is absent or minimized when the transgene inserts in a random location. However, when homologous recombination occurs the endogenous regulatory region are placed in apposition to the incoming transgene, which results in expression of the transgene.

(iii) Site Specific Recombination

Members of the integrase family are proteins that bind to a DNA recognition sequence, and are involved in DNA recognition, synapsis, cleavage, strand exchange, and religation. Currently, the family of integrases includes 28 proteins from bacteria, phage, and yeast which have a common invariant His-Arg-Tyr triad (Abremski and Hoess, 1992). Four of the most widely used site-specific recombination systems for eukaryotic applications include: Cre-loxP from bacteriophage P1 (Austin et al., 1981); FLP-FRT from the 2 μ plasmid of Saccharomyces cerevisiae (Andrews et al., 1986); R-RS from Zygosaccharomyces rouxii (Maeser and Kabmann, 1991) and gin-gix from bacteriophage Mu (Onouchi et al., 1995). The Cre-loxP and FLP-FRT systems have been developed to a greater extent than the latter two systems. The R-RS system, like the Cre-loxP and FLP-FRT systems, requires only the protein and its recognition site. The Gin recombinase selectively mediates DNA inversion between two inversely oriented recombination sites (gix) and requires the assistance of three additional factors: negative supercoiling, an enhancer sequence and its binding protein Fis.

The present invention contemplates the use of the Cre/Lox site-specific recombination system (Sauer, 1993; Gibco/BRL, Inc., Gaithersburg, Md.) to rescue specific genes out of a genome, and to excise specific transgenic constructs from the genome. The Cre (causes recombination)-lox P (locus of crossing-over(x)) recombination system, isolated from bacteriophage P1, requires only the Cre enzyme and its loxP recognition site on both partner molecules (Sternberg and Hamilton, 1981). The loxP site consists of two symmetrical 13 bp protein binding regions separated by an 8 bp spacer region, which is recognized by the Cre recombinase, a 35 kDa protein. Nucleic acid sequences for loxP (Hoess et al., 1982) and Cre (Stemberg et al., 1986) are known. If the two lox P sites are cis to each other, an excision reaction occurs; however, if the two sites are trans to one another, an integration event occurs. The Cre protein catalyzes a site-specific recombination event. This event is bidirectional, i.e., Cre will catalyze the insertion of sequences at a LoxP site or excise sequences that lie between two LoxP sites. Thus, if a construct for insertion also has flanking LoxP sites, introduction of the Cre protein, or a polynucleotide encoding the Cre protein, into the cell will catalyze the removal of the construct DNA. This technology is enabled in U.S. Pat. No. 4,959,317, which is hereby incorporated by reference in its entirety.

An initial in vivo study in bacteria showed that the Cre excises loxP-flanked DNA extrachromosomally in cells expressing the recombinase (Abremski et al., 1983). A major question regarding this system was whether site-specific recombination in eukaryotes could be promoted by a bacterial protein. However, Sauer (1987) showed that the system excises DNA in S. cerevisiae with the same level of efficiency as in bacteria.

Further studies with the Cre-loxP system, in particular the ES cells system in mice, has demonstrated the usefulness of the excision reaction for the generation of unique transgenic animals. Homologous recombination followed by Cre-mediated deletion of a loxP-flanked neo-tk cassette was used to introduce mutations into ES cells. This strategy was repeated for a total of 4 rounds in the same line to alter both alleles of the rep-3 and mMsh2 loci, genes involved in DNA mismatch repair (Abuin and Bradley, 1996). Similarly, a transgene which consists of the 35S promoter/luciferase gene/loxP/35S promoter/hpt gene/loxP (luc⁺hyg⁺) was introduced into tobacco. Subsequent treatment with Cre causes the deletion of the hyg gene (luc⁺hyg^{s}) at 50% efficiency (Dale and Ow, 1991). Transgenic mice which have the Ig light chain κ constant region targeted with a loxP-flanked neo gene were bred to Cre-producing mice to remove the selectable marker from the early embryo (Lakso et al., 1996). This general approach for removal of markers stems from issues raised by regulatory groups and consumers concerned about the introduction of new genes into a population.

An analogous system contemplated for use in the present invention is the FLP/FRT system. This system was used to target the histone 4 gene in mouse ES cells with a FRT-flanked neo cassette followed by deletion of the marker by FLP-mediated recombination. The FLP protein could be obtained from an inducible promoter driving the FLP or by using the protein itself (Wigley et al., 1994).

The present invention also contemplates the use of recombination activating genes (RAG) 1 and 2 to excise specific transgenic constructs from the genome, as well as to rescue specific genes from the genome. RAG-1 (GenBank accession number M29475) and RAG-2 (GenBank accession numbers M64796 and M33828) recognize specific recombination signal sequences (RSSs) and catalyze V(D)J recombination required for the assembly of immunoglobulin and T cell receptor genes (Schatz et al., 1989; Oettinger et al., 1990; Cuomo and Oettinger, 1994). Transgenic expression of RAG-1 and RAG-2 proteins in non-lymphoid cells supports V(D)J recombination of reporter substrates (Oettinger et al., 1990). For use in the present invention, the transforming construct of interest is engineered to contain flanking RSSs. Following transformation, the transforming construct that is internal to the RSSs can be deleted from the genome by the transient expression of RAG-1 and RAG-2 in the transformed cell.

### V. CLINICAL APPLICATION OF HA4-RELATED REAGENTS

It is further contemplated that the HA4 related agents described herein, i.e., HA4 proteins or polypeptides, antibodies raised against such proteins or polypeptides, mutated, truncated or elongated forms of HA4, antibodies raised against such forms, cells engineered to overproduce or lack HA4, proteins that interact with HA4, and agents that stimulate, activate, inhibit or modulate HA4 gene expression may be used to promote or inhibit bone formation. That is, they may be used for the treatment of bone disorders, such as osteoporosis, glucocorticoid induced osteoporosis, Paget's disease, abnormally increased bone turnover, periodontal disease, tooth loss, bone fractures, rheumatoid arthritis, periprosthetic osteolysis, osteogenesis imperfecta, metastatic bone disease, hypercalcemia of malignancy and the like.

A. Screens for Reagents that Modulate HA4 Expression and Function

One may determine whether candidate substances may affect the expression of HA4 by osteoblasts. Cells will be treated with candidate substances either individually or in combination and then examined for HA4 expression at the levels of mRNA, protein, and function. Alternatively, those candidate substances may be tested in vivo by administration to living animals. In one example, osteoblasts will be isolated from those mice after treatment and then examined in vitro for HA4 expression, once again, at the levels of mRNA, protein, and function. In performing these assays, it will be important to also examine the effect(s) of candidate substances on the expression of different isoforms of HA4. In another embodiment, experimental animals will be assessed for in vivo alterations in bone conditions.

Thus, in one embodiment, the present invention is directed to a method for determining the ability of a candidate substance to stimulate the osteoblast activation process, the method including generally the steps of:

(a) providing a composition comprising a population of cells expressing HA4;

(b) incubating the composition with a candidate substance;

(c) assessing HA4 expression or function; and

(d) identifying a candidate substance that modulates HA4 expression or function.

Naturally, one would measure or determine HA4 expression/fucntion composition in the absence of the added candidate substance as a control. A candidate substance which increases the osteoblast development or HA4 expression relative to the activity/expression in its absence is indicative of a candidate substance with stimulatory capability.

It will, of course, be understood that all the screening methods of the present invention are useful in themselves notwithstanding the fact that effective candidates may not be found, since it would be a practical utility to know that HA4 agonists and/or antagonists do not exist. The invention provides methods for screening for such candidates, not in finding them.

Candidate molecules may augment HA4 action without actually affecting HA4 expression or function directly. To test this possibility, test samples will include a suitable cell, HA4 polypeptide or nucleic acids, and a candidate substance. Read out for the assay will be as discussed above.

Any molecule can be a candidate molecule for the purposes of the present invention, for example, from a variety of natural sources. It is envisioned that candidate molecules will be designed and created most effectively using well known combinatorial chemistry techniques, such as those described in VanHijfte et al. (1999) and Floyd et al. (1999), incorporated herein by reference.

B. Therapies Using HA4

As HA4 is involved in bone formation, it may be effectively used for the treatment of bone disorders, such as osteoporosis, glucocorticoid induced osteoporosis, Paget's disease, abnormally increased bone turnover, periodontal disease, tooth loss, bone fractures, rheumatoid arthritis, periprosthetic osteolysis, osteogenesis imperfecta, metastatic bone disease, hypercalcemia of malignancy and the like.

1. Protein Therapy of HA4

A therapy approach is the provision, to a subject, of HA4 polypeptide, active fragments, synthetic peptides, mimetics or other analogs thereof. The protein may be produced by recombinant expression means or, for smaller peptides, generated by a peptide synthesizer. Formulations would be selected based on the route of administration and purpose, including but not limited to liposomal formulations and classic pharmaceutical preparations.

2. Genetic-Based Therapies with HA4

One of the therapeutic embodiments contemplated by the present inventors is the intervention, at the molecular level, in the events involved in the bone formation. Specifically, the present inventors intend to provide, to a bone cell or a precursor cell, an expression construct capable of providing a HA4 polypeptide to that cell. Because the sequence homology between the human and mouse genes, either of these nucleic acids could be used in human therapy, as could any of the gene sequence variants which would encode the same, or a biologically equivalent polypeptide. The lengthy discussion above of expression vectors and the genetic elements employed therein is incorporated into this section by reference. Particularly preferred expression vectors are viral vectors, discussed elsewhere in this document.

Those of skill in the art are well aware of how to apply gene delivery to in vivo and ex vivo situations. For viral vectors, one generally will prepare a viral vector stock. Depending on the kind of virus and the titer attainable, one will deliver 1 to 100, 10 to 50, 100-1000, or up to 1×10⁴, 1×10⁵, 1×10⁶, 1×10⁷, 1×10⁸, 1×10⁹, 1×10¹⁰, 1×10¹¹, or 1×10¹² infectious particles to the patient. Similar figures may be extrapolated for liposomal or other non-viral formulations by comparing relative uptake efficiencies. Formulation as a pharmaceutically acceptable composition is discussed below.

Various routes are contemplated for different disease types. The section below on routes contains an extensive list of possible routes. In a different embodiment, ex vivo gene therapy is contemplated. In an ex vivo embodiment, cells from the patient are removed and maintained outside the body for at least some period of time. During this period, a HA4 gene is delivered to these cells, after which the cells are reintroduced into the patient.

In some embodiments of the present invention a subject is exposed to a viral vector and the subject is then monitored for expression construct-based toxicity, where such toxicity may include, among other things, causing a condition that is injurious to the subject.

3. Pharmaceutical Formulations and Delivery

In a preferred embodiment of the present invention, a method of treatment for a bone disorder by the delivery of an expression construct encoding a HA4 polypeptide is contemplated. Bone disorders, such as osteoporosis, glucocorticoid induced osteoporosis, Paget's disease, abnormally increased bone turnover, periodontal disease, tooth loss, bone fractures, rheumatoid arthritis, periprosthetic osteolysis, osteogenesis imperfecta, metastatic bone disease, hypercalcemia of malignancy and the like may be treated.

An effective amount of the pharmaceutical composition, generally, is defined as that amount sufficient to detectably and repeatedly to ameliorate, reduce, minimize or limit the extent of the disease or its symptoms. More rigorous definitions may apply, including elimination, eradication or cure of disease.

The therapeutic expression construct expressing an HA4 polypeptide may be administered by any of the routes and the route of administration will vary, naturally, with the location and nature of the lesion, and include, e.g., intradermal, transdermal, parenteral, intravenous, intramuscular, intranasal, subcutaneous, percutaneous, intratracheal, intraperitoneal, intratumoral, perfusion, lavage, direct injection, and oral administration and formulation. Treatment regimens may vary as well, and often depend on disease progression, and health and age of the patient. The clinician will be best suited to make such decisions based on the known efficacy and toxicity (if any) of the therapeutic formulations.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of plaque forming units (pfu) for a viral construct. Unit doses range from 10³, 10⁴, 10⁵, 10⁶, 10⁷, 10⁸, 10⁹, 10¹⁰, 10¹¹, 10¹², 10¹³ pfu and higher. Alternatively, depending on the kind of virus and the titer attainable, one will deliver 1 to 100, 10 to 50, 100-1000, or up to about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, or 1×10¹⁵ or higher infectious viral particles (vp) to the patient or to the patient's cells.

Injection of nucleic acid constructs may be delivered by syringe or any other method used for injection of a solution, as long as the expression construct can pass through the particular gauge of needle required for injection. A novel needleless injection system has recently been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermolysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vaccuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The terms “contacted” and “exposed,” when applied to a cell, are used herein to describe the process by which a therapeutic construct encoding a HA4 polypeptide is delivered to a target cell.

C. Diagnostic Applications

In accordance with the present invention, it will also be useful to examine the structure and/or activity of HA4 in cells of a subject. The assays described in the previous section for examining protein levels, mRNA levels, and DNA structure may be applied to the endeavor of examining a clinical sample for defects in HA4. In particular, identification of HA4 in circulation would indicate that serum levels could be used as a diagnostic measure of bone density.

Assays to assess the level of expression of a polypeptide are also well known to those of skill in the art. This can be accomplished also by assaying for HA4 mRNA levels, mRNA stability or turnover, as well as protein expression levels. It is further contemplated that any post-translational processing of HA4 may also be assessed, as well as whether it is being localized or regulated properly. In some cases an antibody that specifically binds HA4 may be used. Assays for HA4 activity also may be used.

1. Northern Blotting Techniques

The present invention therefore employs Northern blotting in assessing the expression of HA4 in a cell such as chrondrogenic cell, osteoblastic cell, or myoblastic cells, but is not limited to such. The techniques involved in Northern blotting are commonly used in molecular biology and are well known to one of skilled in the art. These techniques can be found in many standard books on molecular protocols (e.g., Sambrook et al., 2001). This technique allows for the detection of RNA i.e., hybridization with a labeled probe.

Briefly, RNA is separated by gel electrophoresis. The gel is then contacted with a membrane, such as nitrocellulose, permitting transfer of the nucleic acid and non-covalent binding. Subsequently, the membrane is incubated with, e.g., a chromophore-conjugated probe that is capable of hybridizing with a target amplification product. Detection is by exposure of the membrane to x-ray film or ion-emitting detection devices.

U.S. Pat. No. 5,279,721, incorporated by reference herein, discloses an apparatus and method for the automated electrophoresis and transfer of nucleic acids. The apparatus permits electrophoresis and blotting without external manipulation of the gel and is ideally suited to carrying out methods according to the present invention.

2. Quantitative RT-PCR

The present invention also employs quantitative RT-PCR in assessing the expression or activity of HA4 in a cell such as chrondrogenic cell, osteoblastic cell, or myoblastic cells, but is not limited to such. Reverse transcription (RT) of RNA to cDNA followed by relative quantitative PCR™ (RT-PCR) can be used to determine the relative concentrations of specific mRNA species, such as a HA4 transcript, isolated from a cell. By determining that the concentration of a specific mRNA species varies, it is shown that the gene encoding the specific mRNA species is differentially expressed

In PCR™, the number of molecules of the amplified target DNA increase by a factor approaching two with every cycle of the reaction until some reagent becomes limiting. Thereafter, the rate of amplification becomes increasingly diminished until there is not an increase in the amplified target between cycles. If one plots a graph on which the cycle number is on the X axis and the log of the concentration of the amplified target DNA is on the Y axis, one observes that a curved line of characteristic shape is formed by connecting the plotted points. Beginning with the first cycle, the slope of the line is positive and constant. This is said to be the linear portion of the curve. After some reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR™ is directly proportional to the starting concentration of the target before the PCR™ was begun. By determining the concentration of the PCR™ products of the target DNA in PCR™ reactions that have completed the same number of cycles and are in their linear ranges, it is possible to determine the relative concentrations of the specific target sequence in the original DNA mixture. If the DNA mixtures are cDNAs synthesized from RNAs isolated from different cells, the relative abundances of the specific mRNA from which the target sequence was derived can be determined for the respective tissues or cells. This direct proportionality between the concentration of the PCR™ products and the relative mRNA abundances is only true in the linear range portion of the PCR™ reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR™ products must be sampled when the PCR™ reactions are in the linear portion of their curves.

The second condition that must be met for an RT-PCR study to successfully determine the relative abundances of a particular mRNA species is that relative concentrations of the amplifiable cDNAs must be normalized to some independent standard. The goal of an RT-PCR study is to determine the abundance of a particular mRNA species relative to the average abundance of all mRNA species in the sample. In such studies, mRNAs for -actin, asparagine synthetase and lipocortin II may be used as external and internal standards to which the relative abundance of other mRNAs are compared.

Most protocols for competitive PCR™ utilize internal PCR™ internal standards that are approximately as abundant as the target. These strategies are effective if the products of the PCR™ amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

The discussion above describes the theoretical considerations for an RT-PCR assay for clinically derived materials. The problems inherent in clinical samples are that they are of variable quantity (maling normalization problematic), and that they are of variable quality (necessitating the co-amplification of a reliable internal control, preferably of larger size than the target).

Both of the foregoing problems are overcome if the RT-PCR is performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Other studies are available that use a more conventional relative quantitative RT-PCR with an external standard protocol. These assays sample the PCR™ products in the linear portion of their amplification curves. The number of PCR™ cycles that are optimal for sampling must be empirically determined for each target cDNA fragment. In addition, the reverse transcriptase products of each RNA population isolated from the various tissue samples must be carefully normalized for equal concentrations of amplifiable cDNAs. This is very important since this assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR assays can be superior to those derived from the relative quantitative RT-PCR with an internal standard.

One reason for this is that without the internal standard/competitor, all of the reagents can be converted into a single PCR™ product in the linear range of the amplification curve, increasing the sensitivity of the assay. Another reason is that with only one PCR™ product, display of the product on an electrophoretic gel or some other display method becomes less complex, has less background and is easier to interpret.

3. Immunohistochemistry

The present invention also employs quantitative immunohistochemistry in assessing the expression of HA4 in a cell, tissue or organ sample.

Briefly, frozen-sections may be prepared by rehydrating 50 ng of frozen “pulverized” tumor at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in −70° C. isopentane; cutting the plastic capsule and removing the frozen cylinder of tissue; securing the tissue cylinder on a cryostat microtome chuck; and cutting 25-50 serial sections containing an average of about 500 remarkably intact cell, tissue or organ sample.

Permanent-sections may be prepared by a similar method involving rehydration of the 50 mg sample in a plastic microfuge tube; pelleting; resuspending in 10% formalin for 4 h fixation; washing/pelleting; resuspending in warm 2.5% agar; pelleting; cooling in ice water to harden the agar; removing the tissue/agar block from the tube; infiltrating and embedding the block in paraffin; and cutting up to 50 serial permanent sections.

Other immunohistochemistry techniques that may be employed in the present invention include tissue microarray immunohistochemistry. This method is a recently developed technique that enables the simultaneous examination of multiple tissues sections concurrently as compared to the more conventional technique of one section at a time. This technique is used for high throughput molecular profiling of tumor specimen (Kononen et al., 1998).

4. Western Blotting

The present invention also employs the use of Western blotting (immunoblotting) analysis to assess HA4 activity or expression in a cell such as chrondrogenic cell, osteoblastic cell, or myoblastic cells, but is not limited to such. This technique is well known to those of skill in the art, see U.S. Pat. No. 4,452,901 incorporated herein by reference and Sambrook et al. (2001). In brief, this technique generally comprises separating proteins in a sample such as a cell or tissue sample by SDS-PAGE gel electrophoresis. In SDS-PAGE proteins are separated on the basis of molecular weight, then are transferring to a suitable solid support, (such as a nitrocellulose filter, a nylon filter, or derivatized nylon filter), followed by incubation of the proteins on the solid support with antibodies that specifically bind to the proteins.

5. ELISA

The present invention may also employ the use of immunoassays such as an enzyme linked immunosorbent assay (ELISA) in assessing the activity or expression of HA4 in a cell such as chrondrogenic cell, osteoblastic cell, or myoblastic cells, but is not limited to such. An ELISA generally involves the steps of coating, incubating and binding, washing to remove species that are non-specifically bound, and detecting the bound immune complexes. This technique is well known in the art, for example see U.S. Pat. No. 4,367,110 and Harlow and Lane, 1988.

In an ELISA assay, a HA4 protein sample may be immobilized onto a selected surface, preferably a surface exhibiting a protein affinity such as the wells of a polystyrene microtiter plate. After washing to remove incompletely adsorbed material, it is desirable to bind or coat the assay plate wells with a nonspecific protein that is known to be antigenically neutral with regard to the test antisera such as bovine serum albumin (BSA), casein or solutions of milk powder. This allows for blocking of nonspecific adsorption sites on the immobilizing surface and thus reduces the background caused by nonspecific binding of antisera onto the surface.

After binding of the antigenic material to the well, coating with a non-reactive material to reduce background, and washing to remove unbound material, the immobilizing surface is contacted with the antisera or clinical or biological extract to be tested in a manner conducive to immune complex (antigen/antibody) formation. Such conditions preferably include diluting the antisera with diluents such as BSA, bovine gamma globulin (BGG) and phosphate buffered saline (PBS)/Tween. These added agents also tend to assist in the reduction of nonspecific background. The layered antisera is then allowed to incubate for from 2 to 4 or more hours to allow effective binding, at temperatures preferably on the order of 25° C. to 37° C. (or overnight at 4° C.). Following incubation, the antisera-contacted surface is washed so as to remove non-immunocomplexed material. A preferred washing procedure includes washing with a solution such as PBS/Tween, or borate buffer.

Following formation of specific immunocomplexes between the test sample and the bound antigen, and subsequent washing, the occurrence and even amount of immunocomplex formation may be determined by subjecting same to a second antibody having specificity for the first. To provide a detecting means, the second antibody preferably has an associated enzyme that generates a color development upon incubating with an appropriate chromogenic substrate. Thus, for example, one will desire to contact and incubate the antisera-bound surface with a urease or peroxidase-conjugated anti-human IgG for a period of time and under conditions which favor the development of immunocomplex formation (e.g., incubation for 2 hours at room temperature in a PBS-containing solution such as PBS-Tween).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline-6-sulfonic acid (ABTS) and H₂O₂, in the case of peroxidase as the enzyme label. Quantification is then achieved by measuring the degree of color generation, e.g., using a visible spectra spectrophotometer. The use of labels for immunoassays are described in U.S. Pat. Nos. 5,310,687, 5,238,808 and 5,221,605.

Other immunodetection methods that may be contemplated in the present invention include radioimmunoassay (RIA), immunoradiometric assay, fluoroimmunoassay, chemiluminescent assay, bioluminescent assay. These methods are well known to those of ordinary skill and have been described in Doolittle et al. (1999); Gulbis et al. (1993); De Jager et al. (1993); and Nakamura et al. (1987), each incorporated herein by reference.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Expression of HA4 in Bone

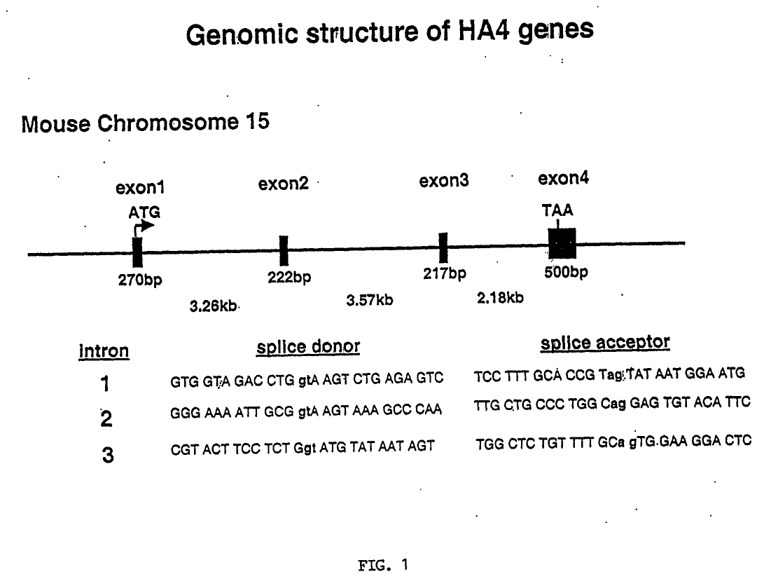

The HA4 gene was isolated from a mouse genomic λ-ZAP library by using HA4 cDNA as a probe. The HA4 cDNA was obtained by a subtraction screening of BMP-untreated and BMP-treated chondrogenic ATDC5 cells, using the Clonetech Subtraction-Suppression Kit. PCR-Amplified cDNAs from ATDC5 cells were subtracted from cDNAs isolated from BMP-treated ATDC5 cells. The structure of the mouse gene for HA4 was found to contain 4 exons and 3 introns. Sizes of exons and introns are indicated in base pairs (FIG. 1). By radiation hybrid mapping, the mouse HA4 gene was mapped to mouse chromosone 15, 8.99 centiRays from D15Mit22. The exon-intron and intron-exon junctions are indicated with the splice donor and splice acceptor sites in small letters. The genomic sequence of the HA4 exons, introns and promoter is provided herein as SEQ ID NO:3. The 2.1 kB promoter that has been used with a β-galactosidase reporter is indicated by underlining. Four exons are indicated in bold; the beginning of the first exon corresponds to the start site of transcription. The double-underlined ATG corresponds to the first methionine residue in HA4. The sequence of HA4 protein was found to be 244 amino acids in length.

Heterozygous HA4 mutant mouse embryo stem (ES) cells were generated by targeted recombination. In the targeting vector the E. coli LacZ gene preceded by an internal ribosomal entry site (IRES) was inserted in exon 2. In addition 67 bp of exon 2 were deleted. Correctly targeted ES cell clones were injected into mouse blastocysts to generate male chimeras, which then produced HA4 heterozygous mutant mice. Homozygous null mutant mice were generated by conventional mating of heterozygous HA4 mutant mice.

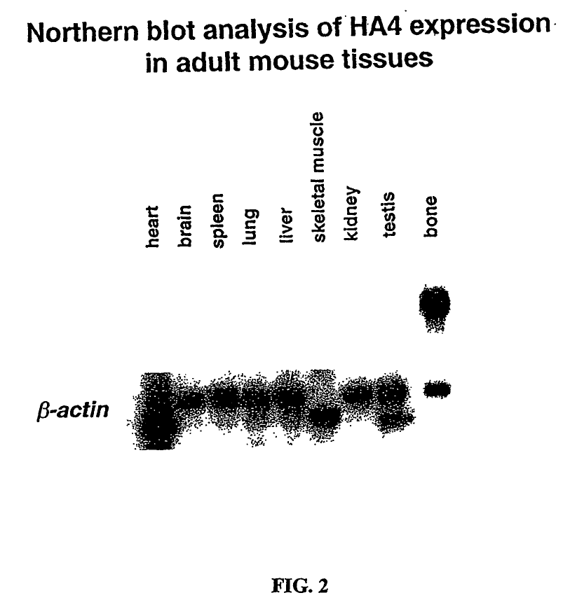

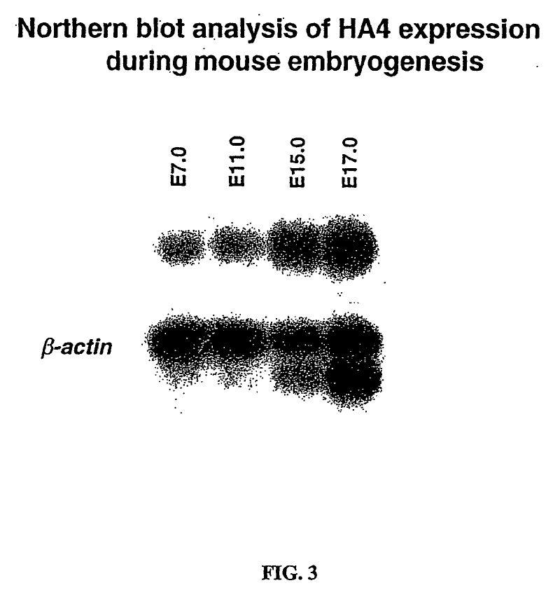

To analyze the expression of HA4 studies were conducted using Northern blotting. Two μg polyA RNAs from different mouse organs was fractionated by electrophoresis in a 1% agarose gel, blotted on a nylon membrane and hybridized with a ³²P-labeled HA4 cDNA probe. The filter was rehybridized with a β-actin cDNA probe to verify equivalent RNA loading. The size of HA4 RNA was found to be approximately 1.6 kb. Expression of HA4 mRNA was also detected using various cell lines. Expression of HA4 was observed in osteoblastic MC3T3-E1 cells and undifferentiated ATDC5 cells, while none of C3H10T1/2 cells, myoblastic C2C12 cells, and Balb/c 3T3 fibroblasts expressed HA4 mRNA in vitro. Moreover, HA4 mRNA was expressed at high levels in bone in adult mice (FIG. 2). In a similar experiment, 2 μg polyA RNA of whole mouse embryos was fractionated by electrophoresis in a 1% agarose gel blotted on a nylon membrane and hybridized with a ³²P-labeled cDNA probe for HA4. The filter was then rehybridized with a β-actin cDNA probe. HA4 expression was analyzed during mouse embryogenesis (FIG. 3).

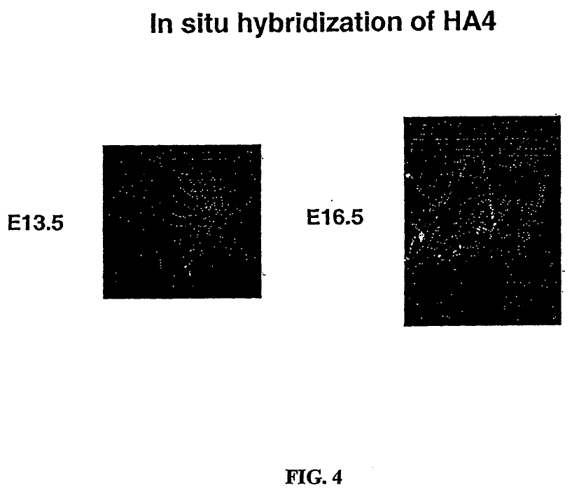

Paraffin sections were generated and hybridized in situ with a ³⁵S-labeled HA4 RNA probe. By in situ hybridization, HA4 expression was found to localize to chondrogenic mesenchymal condensations in E13.5 mouse embryos, and in cartilages, bones, and periosteums in E16.5 mouse embryos. HA4 was detected in mouse embryo forelimb at E13.5 and of mouse embryo elbow at E16.5 (FIG. 4).

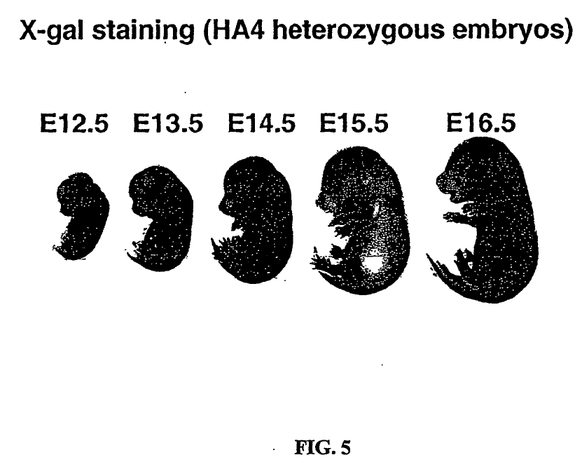

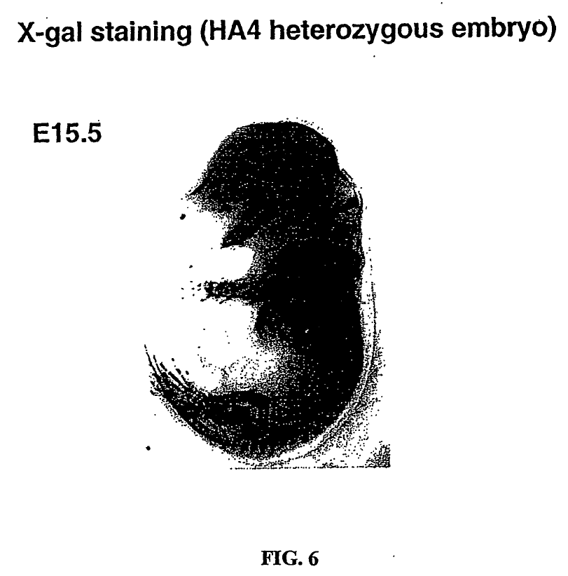

Furthermore, X-gal staining of heterozygous HA4 mutant embryos with a LacZ gene inserted into one HA4 allele revealed specific expression of HA4 in bones and cartilages. HA4 heterozygous mutant embryos at different times of embryonic development were stained with X-gal (FIG. 5). FIG. 6 demonstrates embryonic development in a heterozygous HA4 mutant embryo at day 15.5 by staining with X-gal. The embryo was made translucent by treatment with 0.5 percent KOH and 50 percent glycerol.

Thus, the inventors have identified a secreted polypeptide, HA4, which is expressed selectively in osteoblasts.

### Example 2

### HA4 Deficient Mice

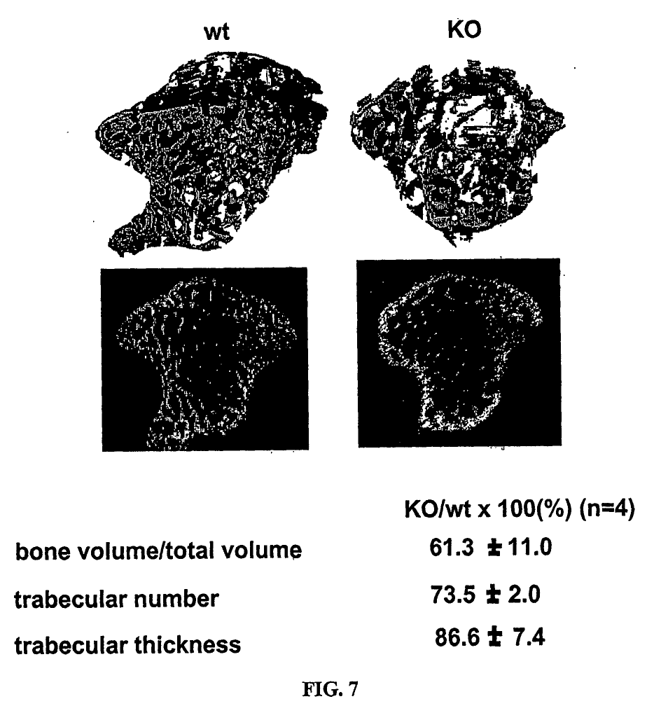

To demonstrate a role for HA4 in bone and cartilage metabolism, the HA4 gene was inactivated in mouse embryonic stem cells. Homologous recombination was used to produce mice that are homozygous-null for HA4. The tibia of 3 month old HA4 null mutant mice was examined by microCT and this analysis compared with that of same sex wild type littermates. The fraction of bone volume over total volume was found to be markedly reduced in HA4-null mutants. This was due both to a decrease in bone trabecular number and to a reduction of trabecular thickness (FIG. 7). Thus, HA4 deficient mice were found to have reduced bone density. The inventors therefore concluded that HA4 is necessary for normal bone density. This phenotype mimics that observed in humans with osteoporosis and provides a model for human osteoporosis.

### Example 3

### Generation of Transgenic Mice and Detection of HA4 Protein in Serum

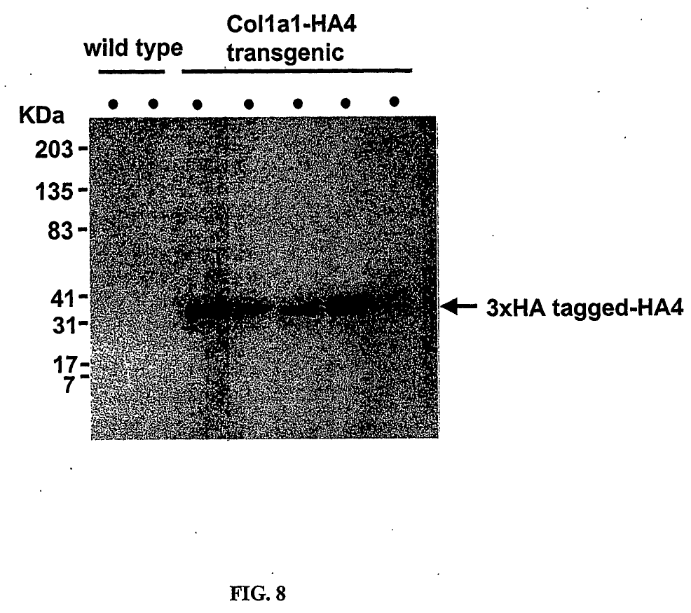

For analyzing the function of HA4 in vivo, transgenic mice were generated in which the HA4 protein is overexpressed in osteoblasts. A recombinant DNA which specifies a HA4 tagged by 3 tandem copies of a short hemaglutinin (HA) peptide was constructed. The DNA for this tagged HA4 was placed under the control of the Col1a1 2.3 kb promoter and transgenic mice were generated that express the tagged HA4 protein in osteoblasts. The 2.3 kb Col1a1 promoter was specifically activated in osteoblasts. Using an antibody against the hemaglutinin peptide, the tagged HA4 protein was detected. The transgenic mice were found to be normal. Immunohistochemistry with rabbit anti-HA antibody showed that 3×HA-tagged HA4 protein is specifically localized in bones of E18.5 mutant embryos. 200 μl of blood were collected from the heart of the transgenic mice, and the serum separated by centrifugation. 3×HA-tagged HA4 protein in the serum (100 μl) was purified with an anti-HA affinity matrix. 3×HA-tagged HA4 protein bound to the matrix was then extracted with Laemli SDS buffer and separated by SDS-PAGE gel. The protein was detected by Western blot using mouse monoclonal anti-HA antibody corresponding to a size of about 35 KDa (FIG. 8). This experiment indicates that the HA4 protein is secreted in the circulation and that the levels of HA4 in serum can be measured.

### Example 4

### Production of Recombinant HA4 Protein

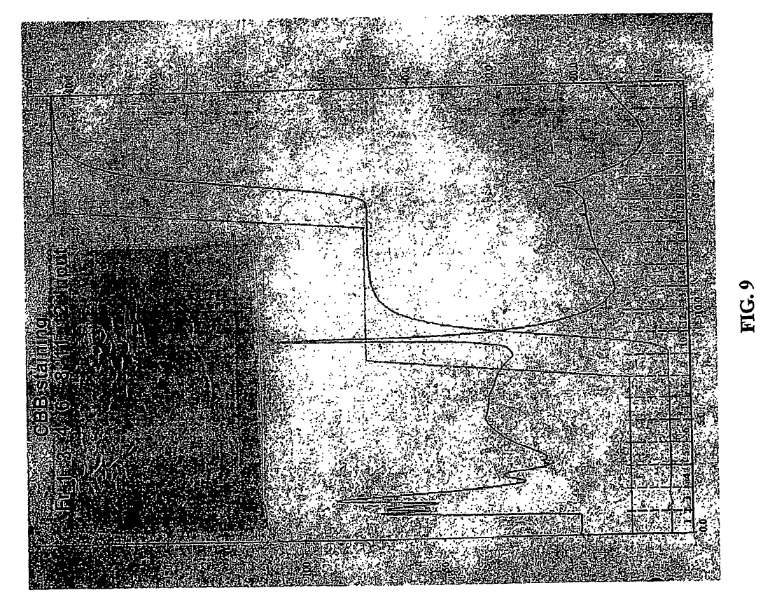

For production of pure recombinant HA4 protein, Flag-tagged, 6×His-tagged mouse HA4 cDNA was cloned into the pBACgus-1 plasmid. This vector was transfected in Sf9 insect cells and produced a high-titer baculovirus stock. For production of recombinant HA4 protein, Sf9 cells were infected with HA4-baculovirus at a multiplicity of infection >5. Twenty-four hours after infection, the conditioned media was collected and the recombinant protein was purified by affinity chromatography with Ni—NTA agarose using a Batch/Gravity-Flow Column purification method. The Ni—NTA agarose bound recombinant protein was eluted with 10 mM Imidazole. The purity of recombinant protein was about 80%. To produce pure recombinant HA4 protein, this purified protein was applied onto a MonoQ column, and pure recombinant HA4 protein eluted with 500 mM NaCl using ACTA System. SDS-PAGE analysis shows essentially 100% purity of this recombinant protein (FIG. 9). This purification scheme can be used to purify homogeneous HA4 and determine the three-dimensional structure of the protein.

### Example 5

### Production of Mouse Monoclonal HA4 Antibody

Due to the inability to obtain a high-titer antibody rabbit anti-HA4 polyclonal peptide antibody and chick anti-HA4 polyclonal peptide antibody could not be produced. One possible reason for this is that HA4 protein exists in serum and is very highly conserved. To resolve this problem, recombinant mouse HA4 protein was injected into HA4 knock-out mice. Five microgram of recombinant HA4 protein was injected into the paw of a knock-out mouse five times every other day. Lymph nodes of inguinal regions were then removed and lymphocytes were prepared. These lymphocytes were fused with mouse myeloma cells, generating hybridomas. Monoclonal antibodies in the conditioned media of these hybridomas were screened by ELISA using recombinant HA4 protein and Western blotting. At least three clones were identified that secrete high-titer monoclonal antibodies. These monoclonal antibodies can be used to measure levels of HA4 in human serum to detect whether changes occur in bone diseases.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VII. REFERENCES

The following references, to the extent that they provide exemplary procedural or other details supplementary to those set forth herein, are specifically incorporated herein by reference.
U.S. Pat. No. 4,196,265
U.S. Pat. No. 4,554,101
U.S. Pat. No. 4,682,195
U.S. Pat. No. 4,683,202
U.S. Pat. No. 4,797,368
U.S. Pat. No. 4,873,191
U.S. Pat. No. 4,959,317
U.S. Pat. No. 5,139,941
U.S. Pat. No. 5,466,468
U.S. Pat. No. 5,846,225
U.S. Pat. No. 5,846,233
U.S. Pat. No. 5,994,136
U.S. Pat. No. 6,013,516
Abremski and Hoess, Protein Eng., 5(1):87-91, 1992.
Abremski et al., Cell, 32:1301-1311, 1983.
Abuin and Bradley, Mol. Cell Biol., 16(4):1851-1856, 1996.
Andrews et al., Mol. Cell Biol., 6(7):2482-2489, 1986.
Angel et al., Cell, 49:729, 1987b.
Angel et al., Mol. Cell. Biol., 7:2256, 1987a.
Antibodies: A Laboratory Manual, Cold Spring Harbor Laboratory, Cold Spring Harbor Press, Cold Spring Harbor, N.Y., 1988.
Atchison and Perry, Cell, 46:253, 1986.
Atchison and Perry, Cell, 48:121, 1987.
Austin et al., Cell, 25(3):729-736, 1981.
Baichwal and Sugden, In: Gene Transfer, Kucherlapati (ed.), New York, Plenum Press, 117-148, 1986.
Banerji et al., Cell, 27(2 Pt 1):299-308, 1981.
Banerji et al., Cell, 33(3):729-740, 1983.
Barribault et al., Mol. Biol. Med., 6:481-492, 1989.
Berkhout et al., Cell, 59:273-282, 1989.
Bi et al., Nat Genet., 22(1), 85-9, 1999.
Bittner et al., Methods in Enzymol., 153:516-544, 1987.
Blanar et al, EMBO J., 8:1139, 1989.
Blomer et al., J. Virol., 71(9):6641-6649, 1997.
Bodine and Ley, EMBO J., 6:2997, 1987.
Boshart et al., Cell, 41:521, 1985.
Bosze et al., EMBO J., 5(7):1615-1623, 1986.
Braddock et al., Cell, 58:269, 1989.
Bradley et al., Bio/Technology, 1992.
Bradley et al., Nature, 309:255-258, 1984.
Brutlag et al, Comput. Appl. Biosci., 6(3):237-245, 1990.
Bulla and Siddiqui, J. Virol., 62:1437, 1986.
Campbell and Villarreal, Mol. Cell. Biol., 8:1993, 1988.
Campbell, On: Monoclonal Antibody Technology, Laboratory Techniques in Biochemistry and Molecular Biology, Burden and Von Knippenberg (Eds.), Elseview, Amsterdam, 13:71-74/75-83, 1984.
Campere and Tilghman, Genes and Dev., 3:537, 1989.
Campo et al., Nature, 303:77, 1983.
Capecchi, Science, 244:1288-1292, 1989.
Capecchi, Trends in Genet., 5:70-76, 1989.
Celander and Haseltine, J. Virology, 61:269, 1987.
Celander et al., J. Virology, 62:1314, 1988.
Chandler et al., Cell, 33:489, 1983.
Chang et al., Mol. Cell. Biol., 9:2153, 1989.
Chatterjee et al., Proc Natl. Acad Sci. U.S.A., 86:9114, 1989.
Choi et al., Cell, 53:519, 1988.
Chou and Fasman, Adv. Enzymol. Relat. Areas Mol. Biol., 47:45-148, 1978.
Chou and Fasman, Annu. Rev. Biochem., 47:251-276, 1978.
Chou and Fasman, Biochemistry, 13(2):211-222; 222-245, 1974.
Chou and Fasman, Biochemistry, 13(2):222-245, 1974.
Chou and Fasman, Biophys. J., 26(3):385-399, 1979.
Cohen et al., J. Cell. Physiol., 5:75, 1987.
Colberre-Garapin et al., J. Mol. Biol., 150:1, 1981.
Costa et al., Mol. Cell. Biol., 8:81, 1988.
Cotton et al., Proc. Natl. Acad. Sci. USA, 89(13):6094-6098, 1992.
Coupar et al., Gene, 68:1-10, 1988.
Cripe et al., EMBO J., 6:3745, 1987.
Culotta and Hamer, Mol. Cell. Biol., 9:1376, 1989.
Cuomo and Oettinger, Nucleic Acids Res., 22(10):1810-1814, 1994.
Curiel, Nat. Immun., 13(2-3):141-164, 1994.
Dale and Ow, Proc. Natl. Acad. Sci. USA, 88(23):10558-10562, 1991.
Dandolo et al., J. Virology, 47:55-64, 1983.
De Villiers et al., Nature, 312(5991):242-246, 1984.
Deschamps et al., Science, 230:1174-1177, 1985.
Edbrooke et al., Mol. Cell. Biol., 9:1908, 1989.
Edlund et al., Science, 230:912-916, 1985.
Evans et al., Nature, 292:154-156, 1981.
Feng and Holland, Nature, 334:6178, 1988.
Fetrow and Bryant, Biotechnology, 11(4):479-484, 1993.
Firak and Subramanian, Mol. Cell. Biol., 6:3667, 1986.
Floyd et al., Prog. Med. Chem., 36:91-168, 1999.
Foecking and Hofstetter, Gene, 45(1):101-105, 1986.
Franceschi, Crit. Rev. Oral Biol. Med., 10(1):40-57, 1999.
Friedmann, Science, 244:1275-1281, 1989.
Frohman et al., Cell, 56:145-147, 1989.
Fujita et al., Cell, 49:357, 1987.
Gilles et al., Cell, 33:717, 1983.
Giphart-Gassler et al., Mutat. Res., 214(2):223-232, 1989.
Gloss et al., EMBO J., 6:3735, 1987.
Godbout et al., Mol. Cell. Biol., 8:1169, 1988.
Goding, 1986, In: Monoclonal Antibodies: Principles and Practice, 2d ed., Academic Press, Orlando, Fla., 60-61; 65-66, and 71-74, 1986.
Goodbourn and Maniatis, Proc. Nat'l Acad. Sci. USA, 85:1447, 1988.
Goodbourn et al., Cell, 45:601, 1986.
Gordon, Intl. Rev. Cytol., 115:171-229, 1989.
Gossler et al., Proc. Natl. Acad. Sci. USA, 83:9065-9069, 1986.
Greene et al., Immunology Today, 10:272, 1989
Grosschedl and Baltimore, Cell, 41:885, 1985.
Grunhaus et al., Sem. in Virol., 200(2):535-546, 1992.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Herr and Clarke, Cell, 45:461, 1986.
Hess et al., J. Adv. Enzyme Reg., 7:149, 1968.
Hirochika et al., J. Virol., 61:2599, 1987.
Hirsch et al., Mol. Cell. Biol., 10:1959, 1990.
Hitzeman et al., J. Biol. Chem., 255:2073, 1980.
Hoess et al., Proc. Natl. Acad. Sci. USA, 79:3398, 1982.
Hogan et al., In: Manipulating the Mouse Embryo: A Laboratory Manual, 2nd ed., Cold Spring Harbor Laboratory Press, 1994.
Holbrook et al., Virology, 157:211, 1987.
Holland et al., Biochemistry, 17:4900, 1978.
Horinouchi et al., Nature Genetics, 10:288-293; 1995.
Horlick and Benfield, Mol. Cell. Biol., 9:2396, 1989.
Horwich et al., Virol., 64:642-650, 1990.
Huang et al., Cell, 27:245, 1981.
Hug et al., Mol. Cell. Biol., 8:3065, 1988.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imler, Vaccine, 13(13):1143-1151, 1995.
Imperiale and Nevins, Mol. Cell. Biol., 4:875, 1984.
Inouye and Inouye, Nucleic Acids Res., 13:3101-3109, 1985.
Jackson Demer, FEBS Lett., 471(1):119-124, 2000.
Jaenisch, Science, 240:1468-1474, 1988.
Jakobovits et al., Mol. Cell. Biol., 8:2555, 1988.
Jameel and Siddiqui, Mol. Cell. Biol., 6:710, 1986.
Jameson and Wolf, Comput. Appl. Biosci., 4(1):181-186, 1988.
Jaynes et al., Mol. Cell. Biol., 8:62, 1988.
Johnson et al., “In: Biotechnology and Pharmacy, Pezzuto et al., Eds., Chapman and Hall, New York, 1993.
Johnson et al., Mol. Cell. Biol., 9:3393, 1989.
Jones, Genetics, 85:12, 1977.
Kadesch and Berg, Mol. Cell. Biol., 6:2593, 1986.
Karin et al., Mol. Cell. Biol., 7:606, 1987.
Karin et al., Mol. Cell. Biol., 7:606, 1987.
Katinka et al., Cell, 20:393, 1980.
Kawamoto et al., Mol. Cell. Biol., 8:267, 1988.
Kelleher and Vos, Biotechniques, 17(6):1110-7, 1994.
Kiledjian et al., Mol. Cell. Biol., 8:145, 1988.
Kim et al., Gene, 103:227-233, 1991.
Kim et al., Nuc. Acids Res., 16:8887-8903, 1988.
Kingsman et al., Gene, 7:141, 1979.
Klamut et al., Mol. Cell. Biol., 10:193, 1990.
Koch et al., Mol. Cell. Biol., 9:303, 1989.
Koller and Smithies, Annu. Rev. Immunol., 10:705-730, 1992.
Kriegler and Botchan, In: Eukaryotic Viral Vectors, Gluzman, ed., Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
Kyte and Doolittle, J. Mol. Biol., 157(1):105-132, 1982.
Larsen et al., Proc Natl. Acad. Sci. USA., 83:8283, 1986.
Lasko et al., Proc. Natl. Acad. Sci. USA, 93(12):5860-5865, 1996.
Laspia et al., Cell, 59:283, 1989.
Latimer et al., Mol. Cell. Biol., 10:760, 1990.
Lavitrano et al., Cell, 57:717-723, 1989.
Lebkowski et al., Mol. Cell. Biol., 8(10):3988-3996, 1988.
Lee et al., Nature, 294:228, 1981.
Lee et al., Nucleic Acids Res., 12:4191-206, 1984.
Levinson et al., Nature, 295:79, 1982.
Lin et al., Mol. Cell. Biol., 10:850, 1990.
Lo, Mol Cell. Biol., 3:1803-1814, 1983.
Lowry et al., Adv. Biochem. Psychopharmacol., 22:119-129, 1980.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Machy et al., Proc. Natl. Acad. Sci. USA, 85(21):8027-8031, 1988.
Majors and Varmus, Proc. Natl. Acad. Sci. USA, 80:5866, 1983.
Mann et al., Cell, 33:153-159, 1983.
Mansour et al., Nature, 336:348-352, 1988.
McLaughlin et al., J. Virol., 62(6):1963-1973, 1988.
McNeall et al., Gene, 76:81, 1989.
Miksicek et al., Cell, 46:203, 1986.
Miller et al., Am. J. Clin. Oncol., 15(3):216-221, 1992.
Mordacq and Linzer, Genes and Dev., 3:760, 1989.
Moreau et al., Nucl. Acids Res., 9:6047, 1981.
Mortensen, Hypertension, 22(4):646-651, 1993.
Muesing et al., Cell, 48:691, 1987.
Mulligan et al., Proc. Natl. Acad. Sci. USA, 78:2072, 1981.
Muzyczka, Curr. Top Microbiol. Immunol., 158:97-129, 1992.
Naldini et al., Science, 272(5259):263-267, 1996.
Ng et al., Nuc. Acids Res., 17:601, 1989.
Nicolas and Rubenstein, In: Vectors: A survey of molecular cloning vectors and their uses, Rodriguez and Denhardt (eds.), Stoneham: Butterworth, pp. 493-513, 1988.
O'Hare et al., Proc. Natl. Acad. Sci. USA, 78: 1527, 1981.
Oettinger et al., Science, 248(4962):1517-1523, 1990.
Ondek et al., EMBO J., 6:1017, 1987.
Onouchi et al., Mol. Gen. Genet., 247(6):653-660, 1995.
Ornitz et al., Mol. Cell. Biol., 7:3466, 1987.
Otterbach and Stoffel, Cell, 81:1053-1061, 1995.
Palmiter et al., Nature, 300:611, 1982.
Paskind et al., Virology, 67:242-248, 1975.
Pech et al., Mol. Cell. Biol., 9:396, 1989.
Perez-Stable and Constantini, Mol. Cell. Biol., 10:1116, 1990.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Queen and Baltimore, Cell, 35:741, 1983.
Quillet et al., J. Immunol., 141(1):17-20, 1988.
Quinn et al., Mol. Cell. Biol., 9:4713, 1989.
Ramirez-Solis et al., Methods Enzymol., 225:855-878, 1993.
Redondo et al., Science, 247:1225, 1990.
Reisman and Rotter, Mol. Cell. Biol., 9:3571, 1989.
Remington's Pharmaceutical Sciences, 15^{th} ed., pages 1035-1038 and 1570-1580, Mack Publishing Company, Easton, Pa., 1980.
Resendez Jr. et al., Mol. Cell. Biol., 8:4579, 1988.
Ridgeway, In: Vectors: A survey of molecular cloning vectors and their uses, Stoneham: Butterworti, pp. 467-492, 1988.
Ripe et al., Mol. Cell. Biol., 9:2224, 1989.
Rittling et al., Nuc. Acids Res., 17:1619, 1989.
Robertson et al., Nature, 322:445-448, 1986.
Rosen et al., Cell, 41:813, 1988.
Roux et al., Proc. Natl. Acad. Sci. USA, 86:9079-9083, 1989.
Sakai et al., Genes and Dev., 2:1144, 1988.
Sambrook et al., In: Molecular cloning, Cold Spring Harbor Laboratory Press, Cold Spring Harbor, N.Y., 2001.
Santerre et al., Gene, 30:147-156, 1984.
Sarkar et al., Cancer Chemother. Pharmacol., 32(6):477-481, 1993.
Satake et al., J. Virology, 62:970, 1988.
Sauer, Methods in Enzymology, 225:890-900, 1993.
Sauer, Mol. Cell. Biol., 7:2087-2096, 1987.
Schaffier et al., J. Mol. Biol., 201:81, 1988.
Schatz et al., Cell, 59:1035-1048, 1989.
Schwartzberg et al., Science, 246(4931):799-803, 1989.
Searle et al., Mol. Cell. Biol., 5:1480, 1985.
Sedivy et al., Proc. Natl. Acad. Sci. USA, 86:227-231, 1989.
Sharp and Marciniak, Cell, 59:229, 1989.
Shaul and Ben-Levy, EMBO J., 6:1913, 1987.
Sherman et al., Mol. Cell. Biol., 9:50, 1989.
Sleigh and Lockett, J. EMBO, 4:3831, 1985.
Small et al., 1985, Mol. Cell Biol., 5:642-648.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Sternberg et al., J. Mol. Bio., 187:197-212, 1986.
Stemberg et al., J. Mol. Bio., 187:197-212, 1986.
Stinchcomb et al., Nature, 282(5734):39-43, 1979.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Szybalska et al., Proc. Natl. Acad. Sci. USA, 48:2026, 1962.
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10:176, 1990.b
Taylor et al., J. Biol. Chem., 264:15160, 1989.
Temin, In: Gene Transfer, Kucherlapati (ed.), New York: Plenum Press, pp. 149-188, 1986.
Thiesen et al., J. Virology, 62:614, 1988.
Thomas and Capecchi, Cell, 51:503-512, 1987.
Thompson et al., Cell, 56:313-321, 1989.
Tratschin et al., Mol. Cell. Biol., 4:2072-2081, 1984.
Treisman, Cell, 42:889, 1985.
Tronche et al., Mol. Biol. Med., 7:173, 1990.
Trudel and Constantini, Genes and Dev., 6:954, 1987.
Tschemper et al., Gene, 10:157, 1980.
Tyndell et al., Nuc. Acids. Res., 9:6231, 1981.
Van der Putten et al., Proc. Natl. Acad. Sci., USA, 82:6148-6152, 1985.
Van Hijftel et al., J. Chromatogr. B. Biomed. Sci. Appl., 725:3-15, 1999.
Vannice and Levinson, J. Virology, 62:1305, 1988.
Vasseur et al., Proc. Natl. Acad. Sci. USA, 77:1068, 1980.
Wagner, EMBO J., 9:3025-3032, 1990.
Wang and Calame, Cell, 47:241, 1986.
Weber et al., Cell, 36:983, 1984.
Weinberger et al. Mol. Cell. Biol., 8:988, 1984.
Weinberger et al., Science, 228:740-742, 1985.
Wigler et al., Cell, 11:223, 1977.
Wigler et al., Proc. Natl. Acad. Sci. USA, 77:3567, 1980.
Willnow and Herz, Methods Cell Biol., 43Pt A:305-334, 1994.
Winoto and Baltimore, Cell 59:649, 1989.
Wolf et al., Comput. Appl. Biosci., 4(1):187-191, 1988.
Wood et al., Proc. Natl. Acad. Sci. USA, 90:4582-4584, 1993.
Yutzey et al. Mol. Cell. Biol., 9:1397, 1989.
Zufferey et al., Nat. Biotechnol., 15(9):871-875, 1997.

1. An isolated nucleic acid segment encoding a polypeptide comprising the sequence as shown in SEQ ID NO:2.

2. The isolated nucleic acid segment of claim 1, wherein the nucleic acid segment comprises the DNA sequence as shown in SEQ ID NO:1.

3. The isolated nucleic acid segment of claim 1, further comprising a promoter operably linked to the region encoding said protein.

4. The isolated nucleic acid segment of claim 3, wherein said promoter is an inducible promoter, a constitutive promoter or a tissue specific promoter.

5. The isolated nucleic acid segment of claim 4, wherein said tissue specific promoter is a bone specific promoter.

6. The isolated nucleic acid segment of claim 1, wherein said nucleic acid segment is comprised within a viral vector.

7. The isolated nucleic acid segment of claim 6, wherein said viral vector is selected from the group consisting of an adenoviral vector, a retroviral vector, an adeno-associated viral vector, a vaccinia viral vector, a herpesviral vector and a pox viral vector.

8. The isolated nucleic acid segment of claim 1, wherein said nucleic acid segment is comprised within a non-viral vector.

9. The isolated nucleic acid segment of claim 8, wherein said non-viral vector is comprised in a lipid carrier.

10. The isolated nucleic acid segment of claim 1, further comprising a region encoding a selectable marker protein.

11. A nucleic acid segment characterized as:
(a) a nucleic acid segment comprising a sequence region that consists of 14 nucleotides that have the same sequence as, or complementary to, at least 14 contiguous nucleotides of SEQ ID NO:1; or
(b) a nucleic acid segment of from 14 to 10,000 nucleotides in length that hybridizes to the nucleic acid segment of SEQ ID NO:1, or the complement thereof, under stringent hybridization conditions.

12. The nucleic acid segment of claim 11, wherein the segment comprises a sequence region of at least 14, at least 17, at least 20, at least 25, or at least 30 contiguous nucleotides from SEQ ID NO:1 or the complement thereof.

13-16. (canceled)

17. The nucleic acid segment of claim 11, wherein the segment is at least 17, at least 20, at least 25 or at least 30 nucleotides in length.

18-20. (canceled)

21. An isolated polypeptide comprising the sequence as shown in SEQ ID NO:2.

22. The isolated polypeptide of claim 21, wherein the polypeptide is comprised in a pharmaceutically acceptable carrier, diluent or excipient.

23. The isolated polypeptide of claim 22, wherein the pharmaceutically acceptable carrier is a lipid carrier.

24. The isolated polypeptide of claim 23, wherein the lipid carrier is a liposome.

25. The isolated polypeptide of claim 23, further comprising a bone tissue targeting agent.

26. A recombinant host cell comprising a nucleic acid segment encoding a polypeptide comprising the sequence as shown in SEQ ID NO:2.

27-32. (canceled)

33. A method of identifying a subject at risk of or suffering from a bone degenerative disease comprising:
(a) obtaining a bone tissue sample from said subject; and
(b) assessing the expression of HA4 in said sample,
wherein a reduced amount of HA4 expression in said sample, as compared to the HA4 expression observed in a healthy subject, indicates that said subject is at risk of or suffers from a bone degenerative disease.

34-35. (canceled)

36. A method of treating a bone degenerative disease in a subject comprising increasing the level or activity of HA4 in bone tissues of said subject.

37-58. (canceled)

59. A method of expressing an HA4 polypeptide in a cell comprising transferring into said cell an expression construct encoding an HA4 under control of a promoter active in said cell, wherein said expression construct effects the expression the HA4 polypeptide.


SEQUENCE LISTING (3 sequences)

SEQ ID NO: 1 - DNA, 735 bp, Artificial Sequence
  CDS (1)..(735)
  Description of Artificial Sequence  Synthetic Primer
atgcacccccaaggccgcgcggcccccccgcagctgctgctcggtctcttccttgtgctgctgctgcttcagttgtccgcaccgtccagcgcctctgagaaccccaaggtgaagcaaaaagcgctgatccggcagagggaggtggtagacctgtataatggaatgtgtctacaaggaccagcaggagttcccggtcgtgatgggagccctggggccaatggcattcctggcacacctggcatcccaggtcgggatggattcaaaggggaaaagggagaatgcttaagggaaagctttgaggagtcctggaccccaaactataagcagtgttcgtggagttcgctgaactatggcatagatcttgggaaaattgcggagtgtacattcacgaagatgcgctccaacagtgctctgcgagttctgttcagtggctcacttcggctcaaatgcaggaatgcatgctgtcagcgctggtattttacatttaatggagctgaatgttcaggacctcttcccatcgaagccatcatctatctggaccaaggaagccctgagttaaattcaactattaatattcatcgtacttcctctgtggaaggactctgtgaagggattggtgctggattggtagatgtggccatctgggttggcacctgttcagattaccccaaaggagacgcttctactggatggaattccgtgtctcgcatcatcattgaagaactaccgaaataa

SEQ ID NO: 2 - protein, 244 aa, Artificial Sequence
  Description of Artificial Sequence  Synthetic Peptide
MHPQGRAAPPQLLLGLFLVLLLLQLSAPSSASENPKVKQKALIRQREVVDLYNGMCLQGPAGVPGRDGSPGANGIPGTPGIPGRDGFKGEKGECLRESFEESWTPNYKQCSWSSLNYGIDLGKIAECTFTKMRSNSALRVLFSGSLRLKCRNACCQRWYFTFNGAECSGPLPIEAIIYLDQGSPELNSTINIHRTSSVEGLCEGIGAGLVDVAIWVGTCSDYPKGDASTGWNSVSRIIIEELPK

SEQ ID NO: 3 - DNA, 20138 bp, Artificial Sequence
  Description of Artificial Sequence  Synthetic Primer
gaattcggatccagacaatgagaaggtaacaatacaaagcttcttttggggaagtccaggtactttattcagttgcaaattagcaaggctcaagctcagtctctggcctcctagtgggccatcttgctctatttttttctttgtgaaaaaacacagactgaacccctaccccagattagaaccggatctggaccctttcaatcattagttagtgggtccttccattttaccatggcataagaattagaagtaactggatgccagnggcaatccgctgcagactgacctttaatatcagtttgcaaaaaatttagaacaaataaggcaaaggcaagatgtgcctttggtgactttgggggatataactgcccctgttttgtctaaaaaggccagaaactctctggaagtggaggctgtgctctaaattaacttgctaggtaattaagaattttagctatctacaatatgaatctctaatattggactaaatattagaccagtccaagattgaataactggtcacactgaagaaaagagaaaaatcattatgactctattaaatgactaatcttactaagtcagattatagtctctgatgttcttttgccataaagttaaaaggcttgaagctagtccaaactagaaaaatggcaagcaaagatgggtctaaaacatgtacccaatttagtttcctagtcacttcaatcagggcactgagtcaactcacctgccagctcatcacacgaattaaccatcatgcttctatagcattctgtggagagaacctatgtttttcccttccctaataaggtagcagtttaggataaaaccataagccaataagtagatctttttaagatactataaagcatttattaaattctttgacatctaaatttcaaaattcaaaaataaaagtatgatttaaatagtgtgcacgggaatgtactaaataatatgcacggggatataatttcccccttctttgtcttttaagaagctaaagttttaaagtttcacaatatctagtccttagaattaataacttgttgacaaaacatctcaaatacttgactgtattcatgtaacaaaggcagtgactaattgcactttcaattgtttttcttaaggagcagttgtaactagaaagcttgaaaagttattaatagtcacatgtgcataatttatttatttactagaaataagagtatttatttgcagcaatttattaagtaaaggtcctcaagaattaacactattatgtggaaacaagtagtaactgaggacactaagaggaaagaattatacatgcacaattttcaagaatactaaaatattatcttaaaccataactgctttgaatatacaaagaacaaggttatatattcaatctatcttatatgtacgacctatagactgcctgagatataaatttagcatggcattgttttaagatgtccaggcaatctagaacaatttattaaatgtctaaaaaccaccttcttaaaaggacagcattttttttctatgagttgtatacgcataagcaattgcaaatttgagaattaatagcatctgagggacgaacaatcttaagtaattgtgagctctgagatataacattgactattaatatacaaattaaggtttgattgttcagcattttaaaacaccatctacagcacacaactagattatctgtgctgaaacgggggggggggactcaaaagaataagcatgtgatctaattcacatgtacttacatctcatataggggttgttttcaaacacaataattgggatgtatgcatgtatgaatttatagaagatacaaaagcatgtataaaaacaatttttaacttatctttgggatattgattgtcaatctatttaaaatgatggtatgtcataggtcaattattaatattttgtaataaccaaataaattgctgtattgaacaaggaacatacacttagtcaggtttgntttttgtttttttgcttttttgntttgtttntttttttttttggnttttcgagacagggtttctctatagcccttggctgtcctggaactcactttgtagaccaggctggcctcgaactcagaaatccacctgcctctgcctcccgagtgctgggatcaaaggcgtgcgccaccacgcccggctattagtcaggtttttaagacacaatttttttttaaaaaaaaaatacacgaatatacattacaattctttattagtaccacagtggttccaataggatgctgaaactgtattttgaggtgaaaaccacctcaatcttggctgcctgccctcggccttcctctcaggcanaagaggctgaaggcancccaatctgttgtagaccanaaaacccntgccgagtcttagtgggaaaaatatggaggctcatggggcaaaggtgaacgcngccctctcctgagttcgctgtaaagccacctgctccttggcggcctcttgtaccactatagctctgggaaactgtgtgtctcaaggccccaccccacagcagccagcagctagctggcttcacatgggtcaatgatcggctgggaggatgagaaaaatgactttacaataatgttttctcttgccaaagaattgttttgagcacagctaatatctaattaccattgattgtaattaataacaattataaaagctttctttattttctgtaaagccttctgtagtaaactaaaaccctaagtaataaaaagatattgcctctgagtcttttgggtgagagcaaggatttaaagtaaacttctcttgagaggcattagctaataaaatattttccacttaggaaacaatatacactgaaagattaaaactcttggcttcttgtatagaagcagaaatgataaaaatttctcacataatgtagatcaatatttagccatactagaggctaaatattccagtngttncanactcactagaagcaaaacctttacaatcattaagangcaaaggagagagaaaaagaaacaaactttcagatctataataactatataaaaggtaggcaagccagattgtaatgccttgataaattatacagtctgattgaccctttataaactctaaatttgaactttattaaaacaacatctggctagatccgtaaaggtgctcttttagctaaaagaatatctcctggtttgcaagacaaaggaaagtcacacaaaccgaacagaagctgctctgagcttagttcctcttgccatgaggacatatattagaattactaagtttcttttgcaccattaactttgggaaagtaaaactccattttaaaacaatttatcactttcggaatcaacattaaaactttactaatacattgcgaaatttggcctgctgtgccacgtgcttgagaagactcctgagttgccaattttaaggctaaccttctgttaccaatgtaacaattatttaatcttaaccaataacttatgagctgattgtgaaaacacgcagagcacattaccaataaaaaaaatgaagcagttacacttagaccaatcagagaatattaatttttctagctacaatcatagaataaaagcactttatcatttgccaaatacattaataacgattgcagagaaccctccaggaaaaaaccatttatcagcttttttaccccaaaaccaagaggctgcagactctcttttttcctataaagaacagtttctccagcaggggccctcctgcggtgtgctggtttcctggttaaagcatgtggccactcttggctttgatgaaccaccagataaagttttttagccatctttattatccaacataaaacatagaacattcattcattctgactggacacgaacatacatgaattgaacatgtgaacggattggtgcacccaaacacaaaaacacaagagggcagagaacttctgctgtaaggcccagagagaacaaagcagcgcacccctgaaatttctctctgcttctgggacattttcctcccacatgatgcagtttctaactgtggccatccacctgattattaaatcccttttattaaaccctttcttttctcacgcctaaaaaaagcaacttcttggagctgcggctaactgcctgataaatttcactgctgaaactaagtgaatttagcgcgcaggtttaacgcagtttcaacttagcccataccaaccttctccggtacgaattttctttaattattttcttcttccatggagctccaaaccagcaatgcttcttccaaatgtcctctgctgtttcccagtcccgcgttggttcgccaatctgttgaggccagccagcggcccacatgtctgggttctagcctgggaggtatcttggaatctggaagagaagagggaactaggcggctcgagagagaatggaacgataatggaaccaagacatcagtctgatcaaggttcaattttactatttggagacactgggttatgaagtagagggaggggcccattcctgccaaatcatccttggagtccagtttcaggtgaccacgtgttggctccggaacagctaggccgcaggtagcagcagtgggagtgacaggctccacccctgatgctctnttagccctaacagtcaaacctgatcagccagatttcaggctgggggggaggttacaatatttgttggtctttttttcatgcagcttttgggggggggttgttttgttttgttttattgagacaggaatttgctgtgtagcctttattgtcttggaatttgctctgtaaactaggctgtccatgaactcacagagatctacctgcctttgcctccctagtgctggggttaaaggtgttcaccaccaccacctagcccataatctcatattctatcaagggatttaggttccataaatgcacttatcataagaggttccaacagatggaacacaactacacagtataaggtggactaatacatgtgtgtccttggtgaagaatcccttatcttatgtcctttcatgtgcttgctttatcaaaacatcctttcacctgtgtctgctttaggataacactccttcacatgtttgccacagcaaagcaccatcagacacgactgactttccaaagaacccttaagtttccacttcagataggcatcttcttctttaggttaagaaaatttttttcctgtcactctgtggaaaatattttctgtgcatttgacctgtgtttcttctccttccttaattcttattttagatttggtcacttcacagtgtcccagatttcctggatgttttgaaccaggagttatttttagattgaacattttctttgacctatgtattcatttcttctaccatgtcttcagtgtctgagattctcttttttaattcttgaattctgttggtgaagcttgcctctgtggttgctgttcaaattcctatattttcatttccatatttccctcagtttggggtctctttattaattctgttggaggaagggggtccaggagtcacctcacaaatcacatattcccaggattgattgatcaggaccaccggccagactcaggagctgaactgtgatgtagagtcaagatcctacagggcttttaaagcctgagagctataataaccatctctgctaagttactccaccaatcataacttagggatagggctttctgtaggagcatgtctttgttgtgtacttattttgctcctattggttagggtattcaactatggcagaggacttgcctcatcttatattcatgtcttagcttgccaaccaggatatcagttttccacatacatgtctctttctgtcaagtaggatatcaagttcccagggaggtcttggaaacctaaactttattctgcccctactcaaaatggaagtcttattctaaataggtacaggtgtctctcttatgttgggatccatcccaagggcagcttaaaaggcaaatactataaaggctgatacacaggtgcagaaagtgttggtttctgagaacatcctagtaacagaagtaacagcatatgagaaagtttccttgtgatattaggaacagcacaaactggtaggttagacgggtaacagttaccaagaccttaacaatcccagttcctctttcaggtcttgagtggctgtattcctttccttccattgtttgtgctttcatagacttctttaagggatttaatgttttctttttaaggacctctagcatacacatataggctgtgttaaggtctttatgtgtgcttccagggtgtaatactcagggcctgctgtgatagggttggtgggttctagtggagacctatcgtcctggctgtaattggttgtgcaggggtagcctgtaggttcccaatgagtgtgtgcctgagctggatgcttgggaaaaacattgagtgacgggaggaaagtggggggccagggatctgtatgcttcactgaagatgggtgcagaagcagcctagggctgagactgaggggttccactctgagaagcagagggagaggtgaagatctgcagttagcccacctgcgtccctgcccagtgtggcctgtgggttcccagggagtgccggctggagttgggggtggagggtaggacagggcaatgagtgggggaagggaatttaggaggggaagatctgtgggatccaccagcgatgaggtggctgtggtggaagccgctgcaggagttagcgcagagctcaggatgaaactagggattgggcgtggaggaatggagggagcgtggaggtcgcctctccctctccctggataggtaggtcacccatttgcttcccgtcagagagtgcctaagagagttggaggctgctttcctggttgaagttggatagaacttttcaaactatattaatctgatgtgaaataagcacgtgaaagtgaacctccagcactgaatgttggctattttctaccagcctcagttcacctatgaatggagactccaggctgcatcgtcccccacaggattgccaagaggccacgtgaacaaagctttacattttggagtttagaagggggtaacactcaaacactatcgattatttgagtcataggactcttatagactgttatattctggctctctccatttaataccccaaatgtcacttttttttttttttttaataatgagccttagttttttaggaatgaaggaacacgaaggtgattcctgaggccgagttaagacacgtgcctctaagaaactcaggagttgcggtctccattcccccaccaccaccacctgtggtttctgaccactgtcaccctgcctggtctctgctttcctctctggttctgcagcaccccgcgggggtctgggcgggcggagctgcggaggaggggcgggctagacccgggacccaggcctataacagtatgcaaagctccccggcgtccagggggtgggagggaaaaaggaggccggcctcaatgaaaggcgcattgatgcggcgggctgcagggctgggccagacgctgagcagggtcaggctcctgccgacccctttacctcctgctccgcgcttcgcagccaccgcacaccatgcacccccaaggccgcgcggcccccccgcagctgctgctcggtctcttccttgtgctgctgctgcttcagttgtccgcaccgtccagcgcctctgagaaccccaaggtgaagcaaaaagcgctgatccggcagagggaggtggtagacctggtaagtctgagagtcggtcctgacctcagtgctggaagagaggactcagccaggatcgcaccggaagggcatcagtatagatggtggtggtgctgaccgtaggggtgagtgtagggcagcacgttaagaagcttgagtgcctcagtgtcctgccttgtgtacctgtgtggggacggatctgacgcacgcctgcagcagagtcttgaaccgctacgggagatcatgagaggtcaccacatgctccgacgtgggtcaggtgggatgcccaaatccgtgtagtcgcccagtaatttctggctccaggggaggccaccgttgggagaagtgggggatgctgtggctgcaactggagtagactgagttagtcagttgatttcaaaagaaagcccgaggaagaccctaggccagctggtcgcttggccctgggccaaggctgtgcaacgtgtcctttgtgaggaccagtggccacgatctgccacgtctgcctggaggagangctaacaacccccacaaagcatttgttcagctaacttgaagattatgaatcactttgtgtcatctccctgggaaatatgaactgcagtttactccttagaggaccacagcttgagccaggagtggtcagagactttgaagctgaaggggaaaaatgaaggccccactaggagcccttccaaggacccatttttgcctgatctgtttaaaacagatgagcagtcaggtcttaacctgtgactgccagtcaggaacactgtactcaagctaaggggaaggaaagcgcttccaggaaagcaaatatcccaagggctttctgagaggctaatctgtgggaaagtctgtttgcttaaaacctttccctctaaaagtcaataaacctagtggagggcagagagtttgtctgtcccactcaagagccagccatcgatagatttgtagtctttggcacatcataaacttctgtccttaaaccaagctatatggttgtcaggcactgcgatacataaaggacaggggacatttacttattttattattattattattattttagatttgaatttcttccactgacattctaagttgagctaataaaccaagctccttgacagctagttctaaactgattcaaaagcactgggggaaaatccctgctgtttcacgcagcagtggtagggttttgttgttttatgctctgatatataattttcctccacaaaagcatactgtgttggagctacagttctattttgagtgcctaagttgttaaaaaaaaaaaagtgccacatgaatgtggctcgtgtgcagtttgcgtattatgaatgtgtagttaagatacataaaatagtcatttccccataaagctagcattttccccctctaaggattatacagtaccacaactctaccccaacttggaaaagcatactgtgctgccaggctcaggtgcatcctgtagattggatttggttctggtgacagaaaaagtccacacagtcattaggaaggtttccacagattctataaagcgactttgtataggcgctttcaaagcgtgtctttcacgctcccactgaattctgccccctggtggccaacacaggaaatggggcgttgggtgagggaatttgagcttccattcacaggttttcattttgttgactttcactaatgattctaaatacctattggaactagcattttaagttaagaaaagacaaacatactctatgtagcatctttcctgagaggaatttagaaattatcaaatcatactagaggaatttacaacaataaatgaaatgttaaagtaaaaaatttaattggaattcattgtgttttgaaaagtctaacatcatccctgtttctatgtgaactaatacaaggataagtgcaggaattgataagcaacgctcaaaatatttccactgtagctcaatggtagagtacttgcctgatatgtgtaaggttctaagttcaagcctcattgatgtaatgtgaacatgcatgctctctctctctctctctctctctctgtgtgtgtgtgtgtgtgtgtgtgtgtgtgtgtgtgtgtgtgtgtgtgtgtgtgtgtgtgttttgaggtgtggtcccatgtatcccagactaggcacaaatttgctgttttgacaggatcctaaactctgggtccctgcctctgcttcccaggggaaagggattacaggcacacgtgtgctaccaactgttaacttcatggcaagaatgcttattatcatcttcattttatttataaggaaatgggcactgaaatgctgagtaaatggcctggtctcatacatctaagacgggatgagtctagtatttaaaaaaaaaaaaaggttctagaatccacgcctttaaattctacagcacacagaaagaaacaacagaacagagaacactagcgcttacagatggtttgctagattattacacacctgctgaggacaggggatcacgtgaaactaactgggccacagactcctcttgaggaagacagaatggactggagaagttctacccactgcactttcctctgcataaaaatgggaagagcagaggaggggcacatccaactttagagatgttgcgatacaagacagctactgactgctaaagttttatttcacaattatttccctagcaattattttagggaaatcactcaaaaggaaaaaaatctatatggccaagtattctcattatattaatatttataccaaaaaagagaaatacccaaaaccaagcattaaaaatcttgtatagtaatgtaaaagttccaatttatggaaccaagtcccttgataaaatatgggattatgggcctggcagtggtggtgcatgcctttctttaatcctagcacttgagaagcagagagagttctgggacagccaggggtacaaggagaaaccctgtctcttaaaaacaaaacaaaacaaaacaaaacaaaacaaaacaacaataacaacaaaccctaaaacaaacaaataaaaagaaagggattgtaagaagctaacatagaatgtcaggttctggaggagaaattttaaacacccttaaaatgagttcgccataataatccattagaacacggacgaggtgggttagagggactctgccagtttattaaaaataaacgcagctgtgatcatttgactctcacatactgtttatgtattcatgactgtatttctttatttacttattttgtactggggatcaaaccttgagcatcctgcatgccaggcaagagttctaccactggactacctccctatgccctacactgagtctgcatgttaagtaatggtactgcccccctttctcctttgcaccgtagtataatggaatgtgtctacaaggaccagcaggagttcccggtcgtgatgggagccctggggccaatggcattcctggcacacctggcatcccaggtcgggatggattcaaaggggaaaagggagaatgcttaagggaaagctttgaggagtcctggaccccaaactataagcagtgttcgtggagttcgctgaactatggcatagatcttgggaaaattgcggtaagtaaagcccaaattataataaagttgaagcaaaatataagagtttgtataagtcattgccaaatttattttttattttgttgttaaaccaaaggaccttaaattaaaagattcaggataggtaatgtgctgactttttacttaaaaattaatttaaaaaaatggtaacgtttcagctaattttataaaggcatttctaagatagataatcactattttataaagcaaacgcaaaaagtatagcttttcttttttcaaattagacaggaaccagtcttgtaaagtaactttaaattaatctaatacatcctgctgtagcttcagagctaaaagtggaagatgaacctaaaaattccacgtgactgggatccctgctacgggaagtccttttgtccttggtcgccgtgctcagcttaaaactgctgaaattaggggaactgaagtcaatgctagtgatttaaaatagtgacgatggtgattctgtaatatttgtgtaaggagaagcacgcaaaatagcatgtcagggagggcattttgatgaatgatccagggactgttgagtggtcctgtgattagtgctttgtctccaagaggcccagggtgtagctgagggggagcatgcggagcctcgtgtgcataaggccctcggctggatgaagtgccattggcgagtgggggggggggggagaagagacagaactgagaaagtccgtgctgtttccgtatcttcttttgatgaccacctctgtttctgcctccttagctataatttggtgcttcatggttctgcttaccatgaccacccggaaaacagaagcttattttcataaaactctaaggttttaagttgtagtatggcgtcatccacgtttttcatctcaacctcttctgctctattttaaaagttcaagaattgccttgattctgtgtgacaggtttttcatctagctaccacattttggttatacacacacacacacacacacatatatatatatatattaaagaaagtgtgatttgaatgatgactgttatttcaacatttgacaatggtggactgccttcttattcacagttattcaatagaagttgccattttccccccaaagctgtccctttatggttcctgcttttttggtttggttttttattttttggtttttggttttttgagacagggtttctctgtgtagccctggctgtcctggaactcactttgtagaccaggctggcctcaaactcagaaatctgcctgcctctgcctcccgagtgctgggattaaaggcgtgcgccaccaccgcccagcggttcctgcattattaaaacaccatgatttttagcagtggttaccaatgaatacggaaatgttctgcaaggaagagattgtaggtcataagccaatgtacagattttgtgaagaccagctagaaggaagaaaacaaggctagctcttattactgtgctgttcaggtcatttgtctactgtgcgctgttgctcaaaagaaagttgagcctggtgtggtgactcagaataaatctcagtattcaggaagtagagaccactggaaacctctggagttgaaggccatccttggctacatagctatttcaaggccatcttgggctactggaaaccccgactaaaacaagaacaaccacaaccaccaccataaccaccacccaaaaggatggagagagtgaacataatccatcccaacagatatccaccaacactctagcatgccagcgccgtgagggcggggcttcctttgtgtgaacttttgtatccaaccgtctaacatagagttgatattcagtgtttttcaatagaaaaaaatacataggtttgttagctaaaagtactgaagactagctgtttttgaagataaataggaagtgaaataaaaccatctaagaaagccagaagttggttttcttggctcttgtatcaatctgataagtaatctttatccctctataaaatttaagaatgttaaagacatgggggagaaggcaggtaaaaattcacagtgtattttcagtctcttattttactctctcctatgccctctccaccaatttctcttctatttcctccccctccccctccccctcccacttcatgtacttccttaaaactcgaccaggtccacttggtgctgtctatgtgtgtctctgggaacatagggagcctctatgggtctcatctctggagaaaacagattttgtctccctagcagccatcagttgctcacagctccacagctaggggtggggctccatgggctcttccccatgcatgctgggagtttggttgattttgtacaggccttgcacgtgtaacctattgtgagttcatatgtgccatggccctgttgtgtctggcaaattctgtttcattgcaggtgtctactacctcacaatcccccccgccccactccccttgtagtctctgaacattgaccagatgtcgagtctctgccttaatctccatctactgcaaaaggagggttctatgatgagggttgaagaaatgtgttaatttataggtattaaaaaagaaaccttaggggccagtttattactatgtccttttaacagaataatagtattactttcttctctaggaccttatgagctggttggatatggattttgggtccagtgaactatagcaggcctgagtttcatttttttggttgaactggcttttaacccaatcactaaatacaactatttaatgaccccctaacacttgagccaccactgcactagtggcaccaccgctatcacagttcacagggtgcccagctgggtaaaacacttcattactttttccttggcagcatgcactgagccttccagcactatgaaagttaaccaggaagggcgaatcttctatgtcactgacagcttaattttccacatcctatgacttaagtatgtgggaccatcagaaataggagtattattcagctgtaaagaaaaaggaaattatgaaattcacacgtaaatgggtggaagctgaaaatattcatttcgagtgagatccctccaaacccaggaagacacagaacgaagcatgttctctctcatgcatggacgccagctttgaagctttagatatgcatatttaaattaggctatagtcatagaagttatcaagttagtaaggggagggggtacaatgcaggcagtgtggaggaggaaagggaattaccagcacaggttagatgggagggcaggagaggggacagtgtctgagcacggatatacaatactgaagacctttgaaaaaagcctactactgcagaagcatcctaaactatgtacatccatatttgtaaaaggagctaaatggtgtttccctagacatcatagactaccaagtaaaaaagtaccagatatggggtacctctttttgagttattgatcagtggagtctcaaaggcctccccaaatttcagactgtggtcattattatgggttagcctccataacttgatggttgaagaaactttttaatcaaaatgaaaaatgtctggctgtcttgctgccctggcaggagtgtacattcacgaagatgcgctccaacagtgctctgcgagttctgttcagtggctcacttcggctcaaatgcaggaatgcatgctgtcagcgctggtattttacatttaatggagctgaatgttcaggacctcttcccatcgaagccatcatctatctggaccaaggaagccctgagttaaattcaactattaatattcatcgtacttcctctggtatgtataatagtggtgtttctgagtgagcctcaaatctgcctaagaggttgtttgatttccactgtcacagtgggtatctaacctgttagaaataaacctctagctggtccatagtcctctagctggtctctcctccctggactgcaattcacataattttacagatctttttttcaaagggtatagatgctagggtgtaatctctgatcaagggactcagaatctctggtgtaaggctccagaatgtttatcttaaaaacaaaataaaacaaagcaaacaacaacaacaacagaaaccgtagttgtgataatatgccccaaaattgaaaatcaatgataagttagaaacggcaagacagccaggaattctatacagcagtggctttggttctcagctttggcaatacaacgggttcatctgagaaacttgaaaaaaagattcctgccctgggtactgctcctccaggaagattcaattaatggggatttgatcattacatcgtttgcattgcagtgaggctgatggtagacccacacctgtctacagtctgttggcttcaggcttaagtagactcatttctagggtataaggggtcaaggccttggggacacttaagtattgctggcatgcattttcagctagcatggtgtagactactagacaatttaagtgggactgtggactcaccacctacctcccactgtagaagaggtctgggtatgctgtagccagggagggcagagtcctttagttctgtattcctgggcctcagttgaactgcattcacttcagctaaggtggaaacctgacagggcacataagtacctcaaagtcaaccaggtaggaaagctaaatagccacggcactacaatgtcaagagccattttcttcaagaatcagccacctccagtaaggaaaggaatcgcactaagcacagacatcgaaagtaatgcgactctctgcctgtttcagtgaccaaccatgaaatctttctttagatgtgagtgaagaactttggtaaattggaatgcaagatgtatgttagaatgtgagagcccagagggtatgcgtaggatacagtatcaaaccaaagcagagcaaaaagcagaacagaaaacagaacaagccaggtgtggtggcgcacacctttaatcccagcacttgggaggcagaggcaggcagatttctgagttcaaggccagcctggtctacaaagtgagttccaggacagccagggctatacagagaaaccctgtctctaaaaaacaaacaaacaaacaacccccccccaataaacaaaaaacaaacaaacaaacaaacaaaaaaccagaacaaaatgtcctctttaatataatcatcctattaaagggtcagtgaggtggcttagcaggtagaaggcactctcccaccccctcagagtctgaggacctgtgtttgctccctgggacccatatggtagaaggagagaaccaacccttgtaatcagctgtcctctaatcttcacatgtgcactgtggcacgtgtgtatccacacctacatatacacactagatagacggtaggtaggtaggtaggtaggtaggtaggtaggtaggtaggtaggtaggtagataggtaggtaggtaggtaggtaggtaggtaggtaggtgcatacatacatacatagatgtaatttttttgccaaaaataagaatctatttaaagtggattcacagattgaagggatggtatgagattaagctagaatttctttttctcatatagaagtcctatcttggatttgaatagcatctagggatccacgttgaaaaggactttctttaaaaagacttgcatatcttgatccaggctctggttcaggtctcttccaaagttgggaagtcttaaaagttggaagtgtaggcttcccatatgtttgggtaaattgttctttggttctaagtatctagaacacgttttggtttgagagcactgcctatagcatagcaatcatggaaatgcctccaaaaatgtctatgtatcaactaaaaaaatgagacctttattacgaaatcatcaggtactagaatttttaattagctttgcattaaacagaattataacctgatttcctatagtaaatgcaaaattagtggttttgctttttgttcttgggttgtgactggctctgttttgcagtggaaggactctgtgaagggattggtgctggattggtagatgtggccatctgggttggcacctgttcagattaccccaaaggagacgcttctactggatggaattccgtgtctcgcatcatcattgaagaactaccgaaataaagcctctgacggtttcagtccctgcctcgttggctttttaaatcaagcccttgagtggttcatttaaatgacatttaagaaatcacttaaatgaagtgctcagctgaatgaaaaagcaaagttaaatatgtttacagaccaaagtgtgatctcacacttaaaaatctagtattaaccattttatttcagccaaagatggtttcaggatttttttttcattattattttttaagcctatatattggaatgccattacagtatttagtatttccttctataacatataaaggttatgtctttgtaaggactgtatagaattattttatatctgttaaataaaatgcttctaaaacctaagtatttgtttattcgtttgattgtgtagccctggctgtcttgaaatctgcactgtagaccaggctggcctcaaattccgagatctgtcaacctttatttcccatgtgctgagattgaaggtgtgcaccaccatgcctagcaactttattatttttaaattgaagattttgccactgaagttgaattcctagtacttaacatatgtgaaattgaacataaatctaaattttaactattcatttagcaatttatgaaattttagcacatatatataacatggttctgtatcttgagatatatgaagacataaggatgtttatctgacaggaatataacagtattttaggaatttttgtctcctttttttttttattggatattttctttgtttatactttaaatgttttcccctttccaggtctccccttcagaaacccccattccatccccctgcttctatgagggtgctccccccacccacccactcaaatcttcctgccccacccagcattccccttaaatgccaaaaacagtcttttaaaagaacagcattttttcttatgagttggatatgtataaacaatttgcaaattttgagaatttcttagttctaaggangaaggaaccaattttttaagccaatttgggaaagctcntggnaaaattactttaatcccttnttaaaatattncccaaatttaaanggcctttggganttantttcaaaacaattttttaaaaaaaataacccgggtnttaacagggnncaacnaaattttaaatttantcntggggccctgggaaanccnnggganggggaaaaccccnnnnnnnnnnnnnnnnnnnnnnnnnnnnnnnnnnnnnnnnnnnnnnnnnnnnnnnnnnnnnnnnnnnnnnnnnnnnnnnnnnnncagtcagnctttttttcaanaagaaagggaaatggaagctaccaataggtatgtatttattagagatacatctagagtctgctttttgtttaattcttcaggaattcaagactttaagtaccctcaaagcttacagtttntacgttacctagcacgttttcagagagcagcacaaaggttaaacaaaccattttacatccatgtcattttgtaagtgcttgactttgctgccaaagtgaatacatgttatctacagaattttattttctattgaaaacagtgcctgtatcccaaggagaattagctctgattattcctgaactcaaacatactctttagttggtttcttttctttcttttttttttttttttgatacagtggaaataaaacaaacaaacaaacaataacaacacacatgcataccttaaaataaaacagtaacaacaacaacaacacacacacacatcccttaaggtcaaaaacttaaataactgggccataaatggatatatactcaagtagaatagcctctcccaaggcatgccagccatcagtggatcccattctggttcacacacctgcttgtgggttggctggccctttatgttcaaaggtgcccccatcctgctctgcctttccttttgccaacctccccaacttgcaccttctcttagtctcggttacttttcaagctccagttcttcacttttatctgccaactccaagaaagaggaatgatgctccttcagagcttgccctctaacttccggttctgtaattcaaatgggattaaagttctcagcggcatcaaccttcaccctactgaaatagttacaggcttgatgacactccttttgagttgctcctgttgaacattcctgcttctcctaagatgtagacacagttctctggcctacctactgtgttctcttttgaccattaaaacctcaacatgtttatagtaaagctccaagagttctcaagttctggtctcagtctttcagcttgctcttccaattatactgataactcgctgctcataacaggccacctctgctaccacccgccatacttctctccaaaataacttccttcaagtccagcccaccgcccttctaaaatattgccccggttccttataagctgtaagtcaccatctctgataccatcaaccaggctgaagtctctagctcttgactagatacttatcaaatatagccttgttcccacaattcagaacgagctcttggtctaaatttcttaccagtctacaatacagggtaagaactggcttctgtttatattaattagcccagctactgcattgcactctttatttggattcatttactccatcaaagtatagcaatgtactttaaattgttgcattttctggcacacactgctggtggacataagcgg